(19) United States
(12) Patent Application Publication
(10) Pub. No.: US 2021/0188787 A1
(43) Pub. Date: Jun. 24, 2021

(54) DOTA COMPOUNDS AND USES THEREOF

(71) Applicant: Memorial Sloan-Kettering Cancer Center, New York, NY (US)

(72) Inventors: Ouathek Ouerfelli, Fort Lee, NJ (US); Guangbin Yang, Forest Hills, NY (US); Travis Jason Hollmann, New York, NY (US)

(73) Assignee: Memorial Sloan-Kettering Cancer Center, New York, NY (US)

(21) Appl. No.: 17/251,038

(22) PCT Filed: Jun. 13, 2019
(86) PCT No.: PCT/US2019/037100

§ 371 Date: Dec. 10, 2020

Related U.S. Application Data
(60) Provisional application No. 62/684,573, filed on Jun. 13, 2018.

(51) Int. Cl. C07D 257/02; G01N 33/542; G01N 33/535

(52) U.S. Cl. CPC C07D 257/02; G01N 33/535; G01N 33/542

(57) ABSTRACT

Provided herein are DOTA-containing compounds (e.g., compounds of Formula (I)) and complexes that are reactive with an enzyme activator (e.g., a peroxidase) and covalently bind a target analyte (e.g., a protein) in a catalyzed reporter deposition assaying method. The DOTA-containing compounds and complexes are advantageous over existing reporter compounds and complexes because the DOTA moiety allows detection of the reporter moiety by use of mass spectrometry imaging microscopy.

### RELATED APPLICATIONS

The present application claims priority under 35 U.S.C. § 119(e) to U.S. provisional application U.S. Ser. No. 62/684,573, filed Jun. 13, 2018, which is incorporated herein by reference.

### BACKGROUND OF THE INVENTION

Catalyzed reporter deposition (CARD) is a method of signal amplification useful in assaying biological samples for analyte detection. See U.S. Pat. Nos. 5,731,158, 5,583,001, and 5,196,306 which are hereby incorporated by reference. The CARD method utilizes an analyte-dependent enzyme activation system (ADEAS) to catalyze the deposition of reporter groups (e.g., fluorescein, biotin) onto a receptor, the receptor being part of or added to a surface in contact with the components of the assay. These enzymatically deposited labels are detected directly or indirectly, resulting in signal amplification and improved detection limits. Peroxidases, such as horseradish peroxidase (HRP) are the preferred enzymes for the method.

In the method, the peroxidase oxidizes a hydrogen-donating moiety of a substrate conjugate comprising a labeled compound. When the enzyme and the substrate react, a reactive intermediate is formed, which binds covalently with electron-rich residues near the receptor of the reactive intermediate. Hydrogen-donating moieties that are reactive with peroxidase enzymes include substituted phenols, such as tyramides, tyramines, and p-hydroxycinnamoyl-containing compounds. Specific reporters attached to the conjugate can be detected by fluorescence or light microscopy at the specific site of covalent attachment.

An ongoing need exists to identify molecules useful in the CARD method that can effectively bind specific target analytes to improve analyte detection.

### SUMMARY OF THE INVENTION

The macrocycle 1,4,7,10-tetraazacyclododecane-1,4,7-tetraacetic acid (DOTA) is a chelating agent that can be employed in constructing compounds useful in the CARD method of detecting analytes in biological samples. In particular, the chelated form of DOTA can serve as a reporter moiety and be linked to reactive molecules (e.g., phenols) to form a compound that can bind to specific biological target molecules for analyte detection. The present disclosure describes DOTA-tyramide, DOTA-tyramine, and DOTA-cinnamamide compounds, salts, and chelate complexes thereof that are useful for site-specific binding and analyte detection. The DOTA containing compounds offer advantages over existing reporter compounds because the DOTA moiety can be detected directly (e.g., by mass spectrometry based imaging microscopy).

In one aspect, provided are compounds of Formula (I):

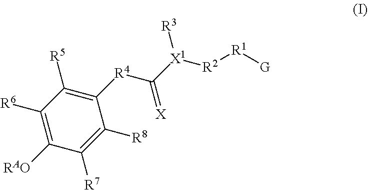

and salts thereof, wherein:

G is a chelating moiety;

R¹ is substituted or unsubstituted alkylene, a bond, —O—, —S—, or —NR^{A}—;

R² is a bond, substituted or unsubstituted carbocyclylene, substituted or unsubstituted heterocyclylene, substituted or unsubstituted arylene, or substituted or unsubstituted heteroarylene;

X¹ is a bond or N;

X is O or S;

R³ is hydrogen, substituted or unsubstituted C₁-C₆ alkyl, or a nitrogen protecting group;

R⁴ is substituted or unsubstituted C₂-C₆ heteroaliphatic, substituted or unsubstituted C₂-C₆ alkylene, substituted or unsubstituted C₂-C₆ alkenylene, or substituted or unsubstituted C₂-C₆ alkynylene;

R⁵ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁶ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁷ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁸ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A}; and each occurrence of R^{A} is independently hydrogen, substituted or unsubstituted acyl, substituted or unsubstituted alkyl, substituted or unsubstituted alkenyl, substituted or unsubstituted alkynyl, substituted or unsubstituted heteroalkyl, a nitrogen protecting group when attached to a nitrogen atom, or an oxygen protecting group when attached to an oxygen atom, or two R^{A} groups are joined to form a substituted or unsubstituted heterocyclic ring.

In certain embodiments, the compound of Formula (I) is a compound of Formula (I-a):

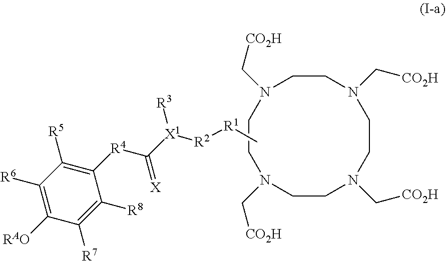

and salts thereof, wherein:

R¹ is substituted or unsubstituted alkylene, a bond, —O—, —S—, or —NR^{A};

R² is substituted or unsubstituted carbocyclylene, substituted or unsubstituted heterocyclylene, substituted or unsubstituted arylene, or substituted or unsubstituted heteroarylene;

X is O or S;

R³ is hydrogen, substituted or unsubstituted C₁-C₆ alkyl, or a nitrogen protecting group;

R⁴ is substituted or unsubstituted C₂-C₆ heteroaliphatic, substituted or unsubstituted C₂-C₆ alkylene, substituted or unsubstituted C₂-C₆ alkenylene, or substituted or unsubstituted C₂-C₆ alkynylene;

R⁵ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁶ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁷ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁸ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A}; and

each occurrence of R^{A} is independently hydrogen, substituted or unsubstituted acyl, substituted or unsubstituted alkyl, substituted or unsubstituted alkenyl, substituted or unsubstituted alkynyl, substituted or unsubstituted heteroalkyl, a nitrogen protecting group when attached to a nitrogen atom, or an oxygen protecting group when attached to an oxygen atom, or two R^{A} groups are joined to form a substituted or unsubstituted heterocyclic ring.

Exemplary compounds of Formula (I) include, but are not limited to:

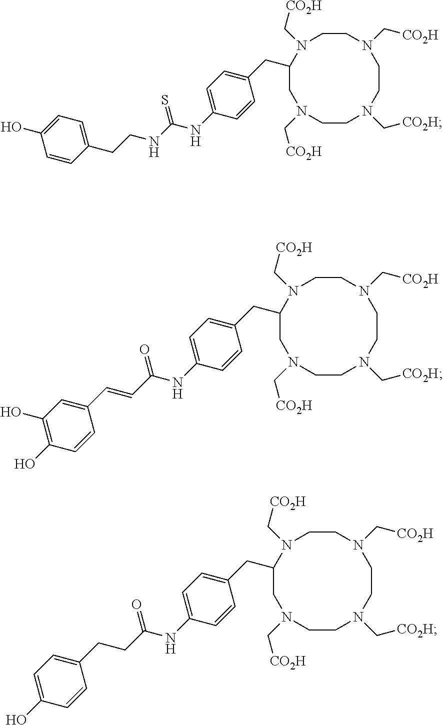 [SMILES: O=C(O)CN1CCN(CC(=O)O)CCN(CC(=O)O)C(Cc2ccc(NC(=S)NCCc3ccc(O)cc3)cc2)CN(CC(=O)O)CC1 ; O=C(O)CN1CCN(CC(=O)O)CCN(CC(=O)O)C(Cc2ccc(NC(=O)/C=C/c3ccc(O)c(O)c3)cc2)CN(CC(=O)O)CC1 ; O=C(O)CN1CCN(CC(=O)O)CCN(CC(=O)O)C(Cc2ccc(NC(=O)CCc3ccc(O)cc3)cc2)CN(CC(=O)O)CC1]

and salts thereof.

In another aspect, provided are salts comprising a compound of Formula (I). In certain embodiments, the salt comprises a metal counterion (e.g., bismuth, lead, yttrium, cadmium, mercury, actinium, thorium, strontium, or a lanthanide). In certain embodiments, the metal counterion is yttrium, praseodymium, or lutetium.

In another aspect, provided are chelate complexes comprising a compound of Formula (I), or a salt of a compound of Formula (I). In certain embodiments, the chelate complex is a trivalent complex.

Exemplary chelate complexes comprising compounds of Formula (I) include, but are not limited to:

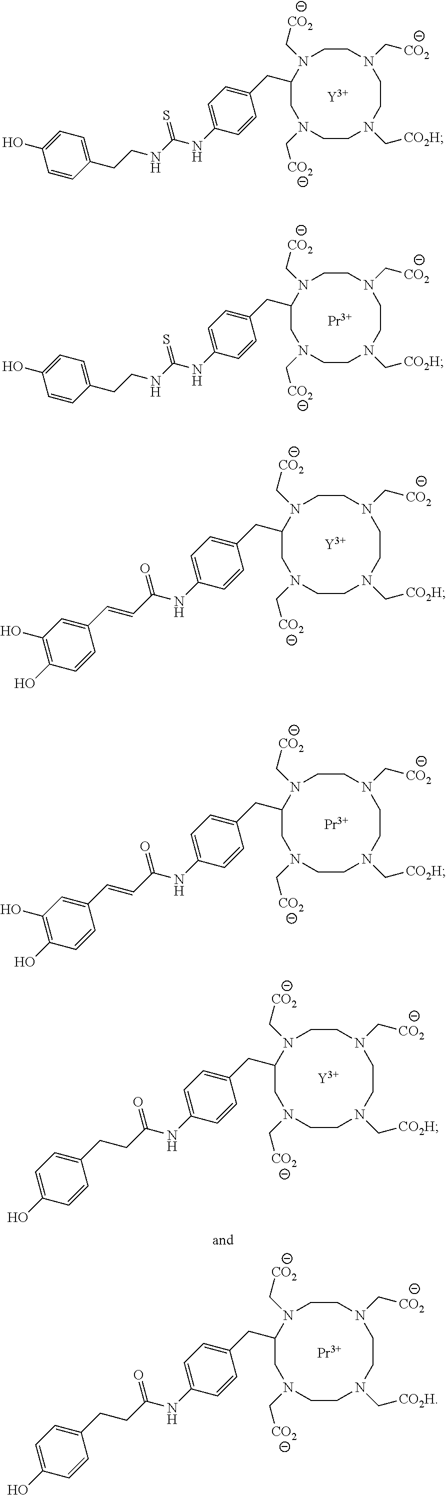

In another aspect, provided are methods of detecting an analyte, the method comprising reacting an enzyme (e.g., a peroxidase) with a chelate complex comprising a compound of Formula (I), or a salt thereof, to form an oxidized chelate complex; contacting the oxidized chelate complex with an biological sample; and detecting the analyte in the biological sample. In certain embodiments, the method further comprises covalent binding of the oxidized chelate complex with the analyte.

In another aspect, provided are kits comprising a compound of Formula (I), or a salt thereof, or a chelate complex comprising a compound of Formula (I). In certain embodiments, the kit further comprises instructions for use.

The details of certain embodiments of the invention are set forth in the Detailed Description of Certain Embodiments, as described below. Other features, objects, and advantages of the invention will be apparent from the Definitions, Examples, and Claims.

### BRIEF DESCRIPTION OF THE DRAWINGS

FIG. 1 is a series of micrographs showing results of CARD assays employing exemplary compounds of the disclosure. The expression of the intermediate filament protein nestin was detected in normal human kidney podocytes using a goat anti-nestin polyclonal antibody with a rabbit anti-goat secondary horseradish peroxidase/3,3′-diaminobenzidine (DAB) chromogenic assay and standard light microscopy (FIG. 1A). The chromogenic signal for nestin expression was not seen with negative control conditions including without anti-nestin primary antibody (FIG. 1C) or without secondary antibody (FIG. 1B). When DAB was replaced with exemplary compounds 1, 2, and 3 complexed to Pr³⁺; (FIG. 1E, FIG. 1I, FIG. 1M) or Y³⁺ (FIG. 1F, FIG. 1J, FIG. 1N), the complex was oxidized by peroxidase and deposited at the site of the anti-nestin antibody. This specific deposition of complex was detected with an anti-DOTA chelate antibody (clone 2D12.5) chromogenic assay using DAB (FIG. 1E, FIG. 1I, FIG. 1M, FIG. 1F, FIG. 1J, FIG. 1N). Empty DOTA was not recognized by the anti-DOTA chelate antibody (clone 2D12.5), therefore chromogenic signal was not seen in these assay conditions (FIG. 1G, FIG. 1K, FIG. 1O). Nestin-specific signal was not seen without anti-DOTA chelate antibody (FIG. 1H, FIG. 1L, FIG. 1P) or without the DOTA chelate complex in the anti-DOTA antibody assay (FIG. 1D).

FIG. 2 is a series of images showing detection of compounds 1 and 3 using a mass spectrometry imaging device. The expression of the cytokeratin 7 (CK7) intermediate filament protein was detected in normal human placenta sections (4 μm; formalin fixation and paraffin embedded) with immunohistochemistry using a mouse anti-CK7 primary antibody followed by an anti-mouse secondary horseradish peroxidase/3,3′-diaminobenzidine (DAB) chromogenic assay and a standard light microscope (H). When DAB was replaced with compound 1 or 3 chelating Pr³⁺ (FIG. 2A, FIG. 2B) or Y³⁺ (FIG. 2C, FIG. 2D), the complex was oxidized by peroxidase and deposited at the site of the anti-CK7 primary antibody. The deposited complexes were detected. Secondary ion mass spectrometry imaging peaks of Pr³⁺ (molecular weight=141 g/mol) and Y³⁺ (molecular weight 88.9=g/mol) containing compounds are shown with adjacent final images from the device (FIGS. 2A-D showing signal in white) comparable to the specific staining of the placental syncytiotrophoblast cells shown with standard DAB staining (FIG. 2H) and highlighted on a hematoxylin and eosin stained section (FIG. 2I). The arrow in FIG. 2I refers to the syncytiotrophoblast layer of placenta. As a control, compounds 1 and 3 not chelated to a metal were not recognized by the mass spectrometry imaging device and therefore specific CK7 signal was not seen using these control assay conditions (FIG. 2E, FIG. 2F). The dilution of the compound is shown parenthetically.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemnistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L., Stereochemistry of Carbon Conpounds (McGraw-Hill, N Y, 1962); and Wilen, S. H., Tables of Resolving Agents and Optical Resolutions, p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

In a formula, [symbol] is a single bond where the stereochemistry of the moieties immediately attached thereto is not specified, [symbol] is absent or a single bond, and [symbol] or [symbol] is a single or double bond.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₃ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C⁵) (e.g., n-pentyl, 3-pentanyl, amyl, neopentyl, 3-methyl-2-butanyl, tertiary amyl), and hexyl (C⁶) (e.g., n-hexyl). Additional examples of alkyl groups include n-heptyl (C₇), n-octyl (C⁸), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and 1 or more heteroatoms within the parent chain (“heteroC₁₋₁₈ alkyl”). In some embodiments, a heteroalkyl group is a saturated group having 1 to 16 carbon atoms and 1 or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀ alkyl. In certain embodiments, the heteroalkyl group is an unsubstituted heteroC₁₋₁₀ alkyl. In certain embodiments, the heteroalkyl group is a substituted heteroC₁₋₂₀ alkyl. In certain embodiments, the heteroalkyl group is an unsubstituted heteroC₁₋₁₀ alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified (e.g., —CH═CHCH₃ or

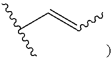

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, N(R^{bb})₃X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R, —SO₂N(R^{bb})₂, —SO₂R^{aa}, —SO₂OR^{aa}, —OSO₂R^{aa}—S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{aa})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl C₆₋₁₄ aryl, and 5-14 membered heteroaryl or two R^{aa} groups are joined to form a 3-14 membered heterocyclyl or 5-14 membered heteroaryl ring, wherein each alkyl, alkenyl alkynyl, heteroalkyl, heteroalkenyl, heteroalkynyl carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion;

each instance of R^{cc} is, independently, selected from hydrogen, C₁₋₁₀ alkyl, C₁₋₁₀ perhaloalkyl, C₂₋₁₀ alkenyl C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl alkynyl, heteroalkyl, heteroalkenyl, heteroalkynyl, carbocyclyl, heterocyclyl aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{se})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb})₂)₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes (—CHO), esters (e.g., —CO₂R^{aa}, —C(═O)SR^{aa}, —C(═S)SR^{aa}), amides (e.g., —C(═O)N(R^{bb})₂, —C(═O)NR^{bb}SO₂R^{aa}, —C(═S)N(R^{bb})₂), and imines (e.g., —C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}—C(═NR^{cc})OR^{aa}, —C(═NR^{cc})N(R^{cc})₂, —SO₂N(R_{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picoiinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacyiamino)acetamide, 3-(p-hydroxyphenyl)propanamide, 3-(o-mtrophenyl)propanamide, 2-methyl-2-(o-nitrophenoxy)propanamide, 2-methyl-2-(o-phenylazophenoxylpropanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenyhnethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsuifonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(l-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylftuorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In certain embodiments, a nitrogen protecting group is benzyl (Bn), tert-butyloxycarbonyl (BOC), carbobenzyloxy (Cbz), 9-flurenylmethyloxycarbonyl (Fmoc), trifluoroacetyl, triphenylmethyl, acetyl (Ac), benzoyl (Bz), p-methoxybenzyl (PMB), 3,4-dimethoxybenzyl (DMPM), p-methoxyphenyl (PMP), 2,2,2-trichloroethyloxycarbonyl (Troc), triphenylmethyl (Tr), tosyl (Ts), brosyl (Bs), nosyl (Ns), mesyl (Ms), triflyl (Tf), or dansyl (Ds).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacohnethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethyisilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl. 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, 1-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). In certain embodiments, an oxygen protecting group is silyl. In certain embodiments, an oxygen protecting group is t-butyldiphenylsilyl (TBDPS), t-butyldimethylsilyl (TBDMS), triisopropylsilyl (TIPS), triphenylsilyl (TPS), triethylsilyl (TES), trimethylsilyl (TMS), triisopropylsiloxymethyl (TOM), acetyl (Ac), benzoyl (Bz), allyl carbonate, 2,2,2-trichloroethyl carbonate (Troc), 2-trimethylsilylethyl carbonate, methoxymethyl (MOM), 1-ethoxyethyl (EE), 2-methyoxy-2-propyl (MOP), 2,2,2-trichloroethoxyethyl, 2-methoxyethoxymethyl (MEM), 2-trimethylsilylethoxymethyl (SEM), methylthiomethyl (MTM), tetrahydropyranyl (THP), tetrahydrofuranyl (THF), p-methoxyphenyl (PMP), triphenylmethyl (Tr), methoxytrityl (MMT), dimethoxytrityl (DMT), allyl, p-methoxybenzyl (PMB), t-butyl, benzyl (Bn), allyl, or pivaloyl (Piv).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻ HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “leaving group” is given its ordinary meaning in the art of synthetic organic chemistry and refers to an atom or a group capable of being displaced by a nucleophile. See, for example, Smith, March's Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifhioromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties. Further exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein).

As used herein, use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and claims. The invention is not intended to be limited in any manner by the above exemplary listing of substituents.

### Other Definitions

The following definitions are more general terms used throughout the present application.

As used herein, the terms “amplify” and “amplification” as used herein means amplification of reporter signal.

As used herein, the term “deposition” means directed binding of a reactive intermediate to the receptor which results from the formation of a covalent bond.

As used herein, the term “reactive intermediate” means the substrate portion of the conjugate has been primed by the enzyme to bind to the receptor. In the compounds of the disclosure, the phenolic moiety is converted to the reactive intermediate, which can bind to the receptor.

As used herein, the term “salt” refers to any and all salts, and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate-ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

### DETAILED DESCRIPTION OF CERTAIN EMBODIMENTS

Provided herein are compounds, salts, and chelate complexes that are useful in methods of catalyzed reporter deposition for detection of analytes in biological samples. In one aspect, the disclosure provides compounds of Formula (I), and salts, chelate complexes, solvates, hydrates, polymorphs, co-crystals, tautomers, stereoisomers, isotopically labeled derivatives, and compositions thereof. In certain embodiments, the disclosure provides chelate complexes comprising the compounds of Formula (I) and a metal cation.

### Compounds

The compounds described herein possess hydrogen donating moieties that can be oxidized when contacted with an enzyme activation system (e.g., a peroxidase). The oxidized compound is useful in methods of catalyzed reporter deposition for the detection of analytes in biological samples. In particular, the macrocyclic DOTA moiety functions as a reporter group which can be detected by a variety of detection methods (e.g., mass spectrometry based imaging). The compounds are particularly useful in the methods and uses described herein when the DOTA moiety comprises a chelate complex having a metal counterion (e.g., lanthanides, yttrium, praesodynium). The DOTA-containing compounds and complexes are advantageous over existing reporter compounds and complexes because the DOTA moiety allows detection of the reporter moiety by use of mass spectrometry imaging microscopy. A compound may be provided for use in any composition, kit, or method described herein as a salt, co-crystal, tautomer, stereoisomer, solvate, hydrate, polymorph, isotopically enriched derivative, or chelate complex thereof.

In one aspect, disclosed is a compound of Formula (I):

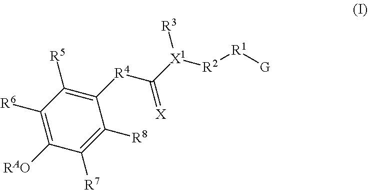

or a salt, co-crystal, tautomer, stereoisomer, solvate, hydrate, polymorph, isotopically enriched derivative, or chelate complex thereof, wherein:

G is a chelating moiety;

R¹ is substituted or unsubstituted alkylene, a bond, —O—, —S—, or —NR^{A}—;

R² is a bond, substituted or unsubstituted carbocyclylene, substituted or unsubstituted heterocyclylene, substituted or unsubstituted arylene, or substituted or unsubstituted heteroarylene;

X¹ is a bond or N;

X is O or S;

R⁵ is hydrogen, substituted or unsubstituted C₁-C₆ alkyl, or a nitrogen protecting group;

R⁴ is substituted or unsubstituted C₂-C₆ heteroaliphatic, substituted or unsubstituted C₂-C₆ alkylene, substituted or unsubstituted C₂-C₆ alkenylene, or substituted or unsubstituted C₂-C₆ alkynylene;

R⁵ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁶ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁷ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};

R⁸ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A}; and

each occurrence of R^{A} is independently hydrogen, substituted or unsubstituted acyl, substituted or unsubstituted alkyl, substituted or unsubstituted alkenyl, substituted or unsubstituted alkynyl, substituted or unsubstituted heteroalkyl, a nitrogen protecting group when attached to a nitrogen atom, or an oxygen protecting group when attached to a nitrogen atom, or two R^{A} groups are joined to form a substituted or unsubstituted heterocyclic ring.

### Group G

Chelation is a type of bonding of ions and molecules to metal ions. It involves the formation or presence of coordinate bonds between a ligand and a single central atom. In certain embodiments, the ligand may be polydentate, or multiply bonded. Typically, these ligands are organic compounds, and are called chelants, chelators, chelating agents, or sequestering agents.

As generally defined herein, G is a chelating moiety. In certain embodiments, G comprises amino acid moieties that act as chelating atoms. In certain embodiments, G is a heterocyclic moiety. In certain embodiments, G is an acyclic moiety. In certain embodiments, G is capable of forming a chelate complex with a metal ion.

In certain embodiments, G is of the formula

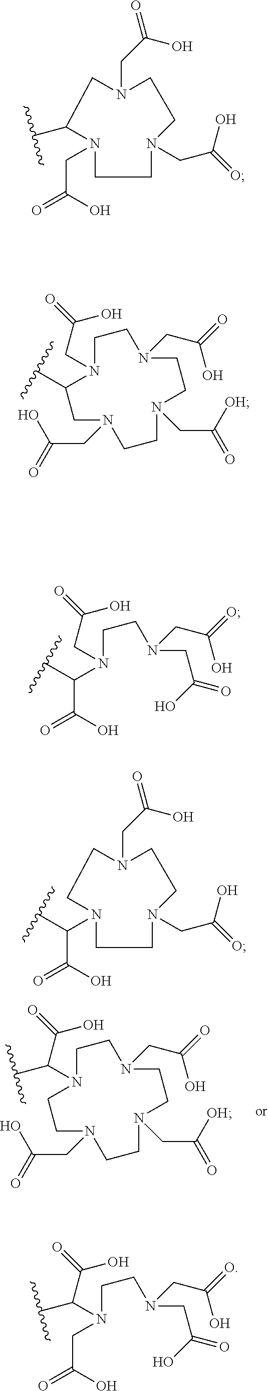

In certain embodiments, G is of the formula

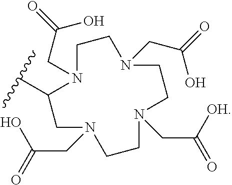 [SMILES: *C1CN(CC(=O)O)CCN(CC(=O)O)CCN(CC(=O)O)CCN1CC(=O)O]

### Group R¹

As generally defined herein, R¹ is substituted or unsubstituted alkylene, a bond, —O—, —S—, or —NR^{A}—. In certain embodiments, R¹ is substituted or unsubstituted alkylene. In certain embodiments, R¹ is substituted or unsubstituted C₁-C₆ alkylene. In certain embodiments, R¹ is unsubstituted C₁-C₆ alkylene. In certain embodiments, R¹ is unsubstituted C₁-C₅ alkylene. In certain embodiments, R¹ is unsubstituted C₁-C₄ alkylene. In certain embodiments, R¹ is unsubstituted C₁-C₃ alkylene. In certain embodiments, R¹ is unsubstituted C₁-C₂ alkylene. In certain embodiments, R¹ is methylene. In certain embodiments, R¹ is ethylene. In certain embodiments, R¹ is propylene. In certain embodiments, R¹ is butylene. In certain embodiments, R¹ is pentylene. In certain embodiments, R¹ is hexylene.

### Group R²

As generally defined herein, R² is a bond, substituted or unsubstituted carbocyclylene, substituted or unsubstituted heterocyclylene, substituted or unsubstituted arylene, or substituted or unsubstituted heteroarylene. In certain embodiments, R² is substituted or unsubstituted carbocyclylene, substituted or unsubstituted heterocyclylene, substituted or unsubstituted arylene, or substituted or unsubstituted heteroarylene. In certain embodiments, R² is substituted or unsubstituted carbocyclylene. In certain embodiments, R² is substituted or unsubstituted heterocyclylene. In certain embodiments, R² is substituted or unsubstituted arylene or substituted or unsubstituted heteroarylene. In certain embodiments, R² is substituted or unsubstituted arylene. In certain embodiments, R² is unsubstituted arylene. In certain embodiments, R² is substituted or unsubstituted phenylene. In certain embodiments, R² is unsubstituted phenylene.

In certain embodiments, R¹ is substituted or unsubstituted C₁-C₆ alkylene and R² is substituted or unsubstituted phenylene. In certain embodiments, R¹ is unsubstituted C₁-C₆ alkylene and R² is unsubstituted phenylene.

### Groups X and X¹

As generally defined herein, X is O or S. In certain embodiments, X is O. In certain embodiments, X is S.

In certain embodiments, R¹ is substituted or unsubstituted G-G alkylene; R² is substituted or unsubstituted phenylene; and X is O. In certain embodiments, R¹ is unsubstituted C₁-C₆ alkylene; R² is unsubstituted phenylene; and X is O.

In certain embodiments, R¹ is substituted or unsubstituted C₁-C₆ alkylene; R² is substituted or unsubstituted phenylene; and X is S. In certain embodiments, R¹ is unsubstituted C₁-C₆ alkylene; R² is unsubstituted phenylene; and X is S.

As generally defined herein, X¹ is a bond or N. In certain embodiments, X¹ is A bond. In certain embodiments, X¹ is N.

### Group R³

As generally defined herein, R³ is hydrogen, substituted or unsubstituted C₁-C₆ alkyl, or a nitrogen protecting group. In certain embodiments, R³ is substituted or unsubstituted C₁-C₆ alkyl. In certain embodiments, R³ is unsubstituted C₁-C₆ alkyl. In certain embodiments, R³ is unsubstituted C₁-C₂ alkyl. In certain embodiments, R³ is unsubstituted C₁-C₄ alkyl. In certain embodiments, R² is unsubstituted C₁-C₃ alkyl. In certain embodiments, R³ is unsubstituted C₁-C₂ alkyl. In certain embodiments, R³ is methyl. In certain embodiments, R³ is ethyl. In certain embodiments, R³ is propyl. In certain embodiments, R³ is butyl. In certain embodiments, R³ is pentyl. In certain embodiments, R³ is hexyl. In certain embodiments, R³ is a nitrogen protecting group. In certain embodiments, R³ is hydrogen.

In certain embodiments, R¹ is substituted or unsubstituted C₁-C₆ alkylene; R² is substituted or unsubstituted phenylene; and X is O. In certain embodiments, R¹ is unsubstituted C₁-C₆, alkylene; R² is unsubstituted phenylene; X is O; and R² is hydrogen.

In certain embodiments, R¹ is substituted or unsubstituted C₁-C₆ alkylene; R² is substituted or unsubstituted phenylene; and X is S. In certain embodiments, R¹ is unsubstituted C₁-C₆ alkylene; R² is unsubstituted phenylene; X is S; and R³ is hydrogen.

### Group R⁴

As generally defined herein, R⁴ is substituted or unsubstituted C₂-C₆ heteroaliphatic, substituted or unsubstituted C₂-C₆ alkylene, substituted or unsubstituted C₁-C₆ alkenylene, or substituted or unsubstituted C₂-C₆ alkynylene. In certain embodiments, R⁴ is substituted or unsubstituted C₂-C₆ heteroaliphatic, substituted or unsubstituted C₁-C₆ alkylene, or substituted or unsubstituted C₂-C₆ alkenylene. In certain embodiments, R⁴ is unsubstituted C₂-C₆ heteroaliphatic, unsubstituted C₂-C₆ alkylene, or unsubstituted C₂-C₆ alkenylene. In certain embodiments, R⁴ is unsubstituted C₂-C₆ heteroalkylene, unsubstituted C₂-C₀ alkylene, or unsubstituted C₂-C₆ alkenylene.

In certain embodiments, R⁴ is unsubstituted C₂-C₆ alkylene. In certain embodiments, R⁴ is unsubstituted C₂-C₅ alkylene. In certain embodiments, R⁴ is unsubstituted C₂-C₄ alkylene. In certain embodiments, R⁴ is unsubstituted C₂-C₃ alkylene. In certain embodiments, R⁴ is unsubstituted ethylene.

In certain embodiments, R⁴ is unsubstituted C₂-G, alkenylene. In certain embodiments, R⁴ is unsubstituted C₂-C₅ alkenylene. In certain embodiments, R⁴ is unsubstituted C₂-C₄ alkenylene. In certain embodiments, R⁴ is unsubstituted C₂-C₃ alkenylene. In certain embodiments, R⁴ is unsubstituted ethenylene.

In certain embodiments, R⁴ is substituted or unsubstituted C₂-C₆ heteroalkylene. In certain embodiments, R⁴ is unsubstituted C₂-C₆ heteroalkylene. In certain embodiments, R⁴ is unsubstituted C₂-C₅ heteroalkylene. In certain embodiments, R⁴ is unsubstituted C₂-C₄ heteroalkylene. In certain embodiments, R⁴ is unsubstituted C₂-C₃ heteroalkylene. In certain embodiments, R⁴ is unsubstituted C₂ heteroalkylene. In certain embodiments, R⁴ is of the formula

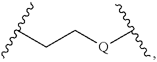

wherein Q is O, NR²⁰, S, S(O), or S(O)₂, and R²⁰ is hydrogen or C₆-C₆ alkyl. In certain embodiments, R⁴ is

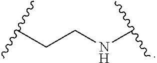 [SMILES: *CCN*]

In certain embodiments, R⁴ is

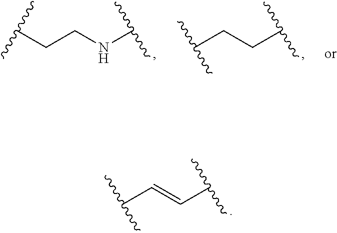

Groups R⁵-R⁸

As generally defined herein, R⁵, R⁶, R⁷, and R⁸ are each independently hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A}.

In certain embodiments, R⁵, R⁶, R⁷, and R⁸ are independently hydrogen, halogen, nitro, cyano, substituted or unsubstituted alkyl, substituted or unsubstituted alkenyl, substituted or unsubstituted alkynyl, substituted or unsubstituted acyl, —N(R^{A})₂, or —OR^{A}. In certain embodiments, R⁵, R⁶, R⁷, and R⁸ are independently hydrogen, halogen, nitro, cyano, unsubstituted alkyl, unsubstituted alkenyl, —N(R^{A})₂, or —OR^{A}. In certain embodiments, R⁵, R⁶, R⁷, and R⁸ are independently hydrogen, halogen, nitro, cyano, unsubstituted C₁-C₆ alkyl, unsubstituted C₂-C₄ alkenyl, —N(R^{A})₂, or —OR^{A}.

In certain embodiments, R⁵, R⁶, R⁷, and R⁸ are independently hydrogen, —N(R^{A})₂, or —OR^{A} In certain embodiments, R⁵, R⁶, R⁷, and R⁸ are independently hydrogen, —N(R^{A})₂, or —OR^{A}; and each R^{A} is independently hydrogen, substituted or unsubstituted acyl, substituted or unsubstituted alkyl, a nitrogen protecting group, or an oxygen protecting group. In certain embodiments, R⁵, R⁶, R⁷, and R⁸ are independently hydrogen, —N(R^{A})₂, or —OR^{A}; and each R^{A} is independently hydrogen, substituted or unsubstituted acyl, a nitrogen protecting group, or an oxygen protecting group. In certain embodiments, R⁵, R⁶, R⁷, and R⁸ are independently hydrogen, —N(R^{A})₂, or —OR^{A}; and each R^{A} is independently hydrogen, a nitrogen protecting group, or an oxygen protecting group. In certain embodiments, R⁵, R⁶, R⁷, and R⁸ are independently hydrogen, —N(R^{A})₂, or —OR^{A}; and each R^{A} is hydrogen. In certain embodiments, R⁵, R⁶, R⁷, and R⁸ are independently hydrogen or —OR^{A}; and R^{A} is hydrogen.

In certain embodiments, at least one of R⁵, R⁶, R⁷, and R⁸ is —OR^{A}. In certain embodiments, at least one of R⁵, R⁶, R⁷, and R⁸ is —OH. In certain embodiments, two of R⁵, R⁶, R⁷, and R⁸ are —OR^{A}. In certain embodiments, two of R⁵, R⁶, R⁷, and R⁸ are —OH. In certain embodiments, R⁵ and R⁸ are hydrogen; and R⁶ and R⁷ are independently hydrogen or —OR^{A}. In certain embodiments, R⁵, R⁷, and R⁸ are each hydrogen. In certain embodiments, R⁵, R⁷, and R⁸ are each hydrogen; and R⁶ is hydrogen or —OR^{A}. In certain embodiments, R⁵, R⁷, and R⁸ are each hydrogen; and R⁶ is hydrogen or —OH. In certain embodiments, R⁵, R⁶, R⁷, and R⁸ are each hydrogen.

### Embodiments of Formula (I)

In certain embodiments, the compound of Formula (I) is a compound of Formula (I-a):

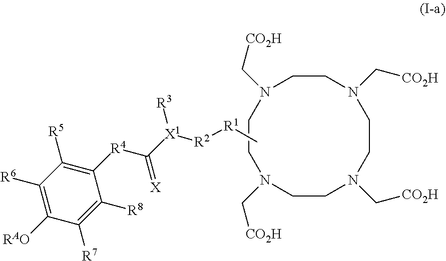

or a salt, co-crystal, tautomer, stereoisomer, solvate, hydrate, polymorph, isotopically enriched derivative, or chelate complex thereof, wherein R¹, R², R³, X, R⁴, R⁵, R⁶, R⁷, R⁸, and R^{A} are as defined herein.

In certain embodiments, the compound of Formula (I) is a compound of Formula (I-b):

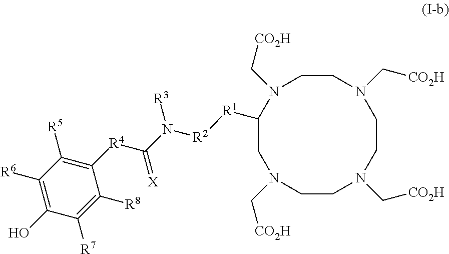

or a salt, co-crystal, tautomer, stereoisomer, solvate, hydrate, polymorph, isotopically enriched derivative, or chelate complex thereof, wherein R¹, R², R³, X, R⁴, R⁵, R⁶, R⁷, R⁸, and R^{A} are as defined herein.

In certain embodiments, the compound of Formula (I) is a compound of Formula (I-c):

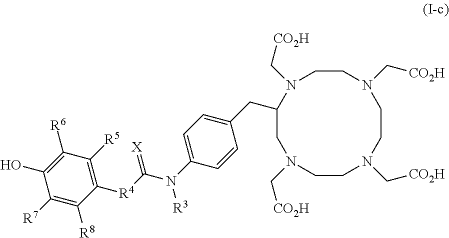

or a salt, co-crystal, tautomer, stereoisomer, solvate, hydrate, polymorph, isotopically enriched derivative, or chelate complex thereof, wherein R³, X, R⁴, R⁵, R⁶, R⁷, R⁸, and R^{A} are as defined herein.

In certain embodiments, the compound of Formula (I) is a compound of Formula (I-d):

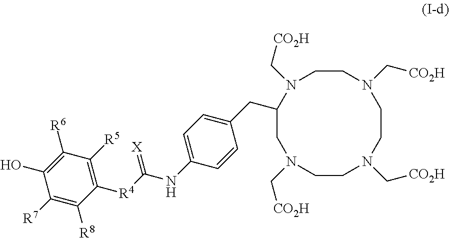

or a salt, co-crystal, tautomer, stereoisomer, solvate, hydrate, polymorph, isotopically enriched derivative, or chelate complex thereof, wherein R⁴, R⁵, R⁶, R⁷, R⁸, and R^{A} are as defined herein.

In certain embodiments, the compound of Formula (I) is a compound of Formula (I-e):

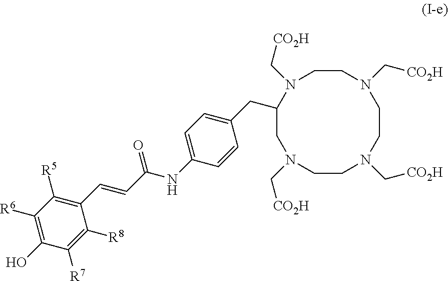

or a salt, co-crystal, tautomer, stereoisomer, solvate, hydrate, polymorph, isotopically enriched derivative, or chelate complex thereof, wherein R⁵, R⁶, R⁷, R⁸, and R^{A} are as defined herein.

In certain embodiments, the compound of Formula (I) is a compound of Formula (I-f):

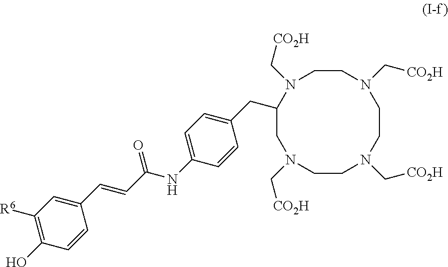

or a salt, co-crystal, tautomer, stereoisomer, solvate, hydrate, polymorph, isotopically enriched derivative, or chelate complex thereof, wherein R⁶ and R^{A} are as defined herein.

In certain embodiments, the compound of Formula (I) is a compound of Formula (I-g):

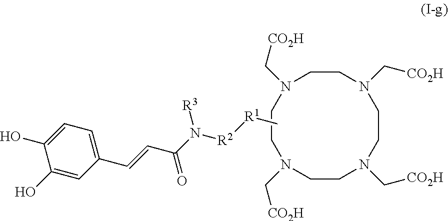

or a salt, co-crystal, tautomer, stereoisomer, solvate, hydrate, polymorph, isotopically enriched derivative, or chelate complex thereof, wherein R¹, R², and R³ are as defined herein.

In certain embodiments, the compound of Formula (I) is a compound of Formula (I-h):

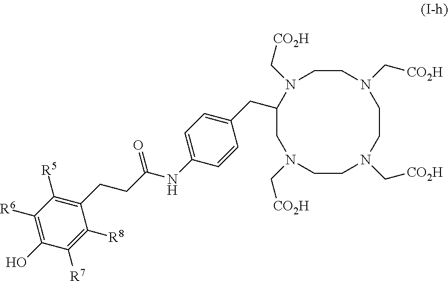

or a salt, co-crystal, tautomer, stereoisomer, solvate, hydrate, polymorph, isotopically enriched derivative, or chelate complex thereof, wherein R⁵, R⁶, R⁷, R⁸, and R^{A} are as defined herein.

In certain embodiments, the compound of Formula (I) is a compound of Formula (I-i):

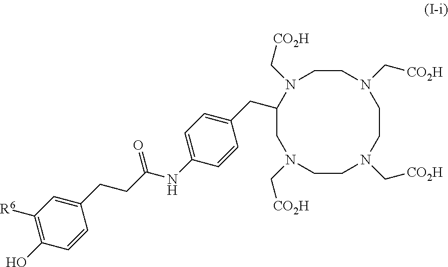

or a salt, co-crystal, tautomer, stereoisomer, solvate, hydrate, polymorph, isotopically enriched derivative, or chelate complex thereof, wherein R⁶ and R^{A} are as defined herein.

In certain embodiments, the compound of Formula (I) is a compound of Formula

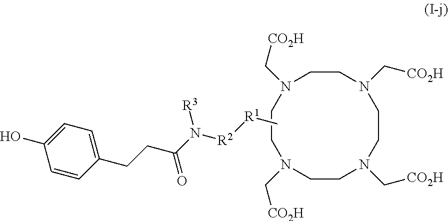

or a salt, co-crystal, tautomer, stereoisomer, solvate, hydrate, polymorph, isotopically enriched derivative, or chelate complex thereof, wherein R¹, R², and R³ are as defined herein.

In certain embodiments, the compound of Formula (I) is a compound of Formula (I-k):

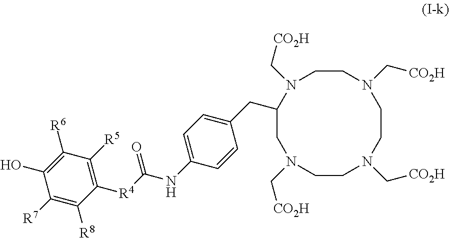

or a salt, co-crystal, tautomer, stereoisomer, solvate, hydrate, polymorph, isotopically enriched derivative, or chelate complex thereof, wherein R⁴, R⁵, R⁶, R⁷, R⁸, and R^{A} are as defined herein.

In certain embodiments, the compound of Formula (I) is a compound of Formula (I-l):

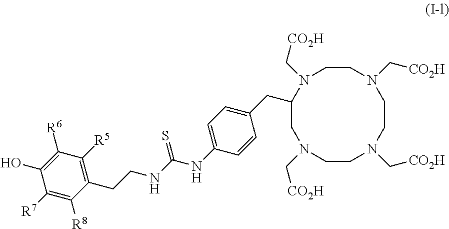

or a salt, co-crystal, tautomer, stereoisomer, solvate, hydrate, polymorph, isotopically enriched derivative, or chelate complex thereof, wherein R⁵, R⁶, R⁷, R⁸, and R^{A} are as defined herein.

In certain embodiments, the compound of Formula (I) is a compound of Formula (I-m):

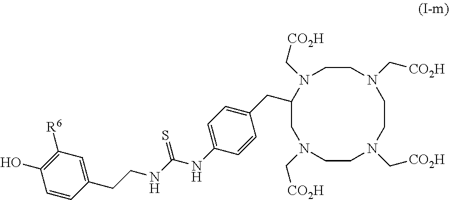

or a salt, co-crystal, tautomer, stereoisomer, solvate, hydrate, polymorph, isotopically enriched derivative, or chelate complex thereof, wherein R⁶ and R^{A} are as defined herein.

In certain embodiments, the compound of Formula (I) is a compound of Formula (I-n):

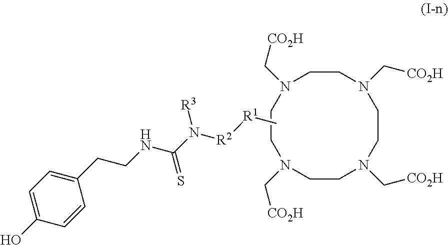

or a salt, co-crystal, tautomer, stereoisomer, solvate, hydrate, polymorph, isotopically enriched derivative, or chelate complex thereof, wherein R¹, R², and R³ are as defined herein.

In certain embodiments, the compound of Formula I is a compound of Table 1, or a salt, co-crystal, tautomer, stereoisomer, solvate, hydrate, polymorph, isotopically enriched derivative, or chelate complex thereof.

TABLE 1

| | |
|---|---|
| 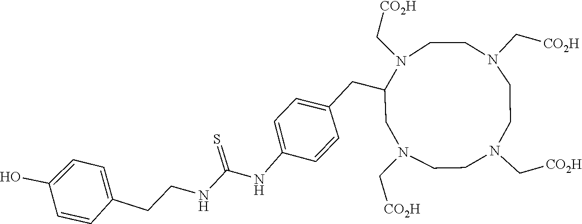 [SMILES: O=C(O)CN1CCN(CC(=O)O)CCN(CC(=O)O)C(Cc2ccc(NC(=S)NCCc3ccc(O)cc3)cc2)CN(CC(=O)O)CC1] | (1) |
| 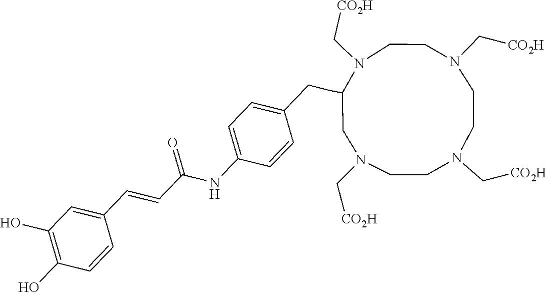 [SMILES: O=C(O)CN1CCN(CC(=O)O)CCN(CC(=O)O)C(CCc2ccc(NC(=O)/C=C/c3ccc(O)c(O)c3)cc2)CN(CC(=O)O)CC1] | (2) |
| 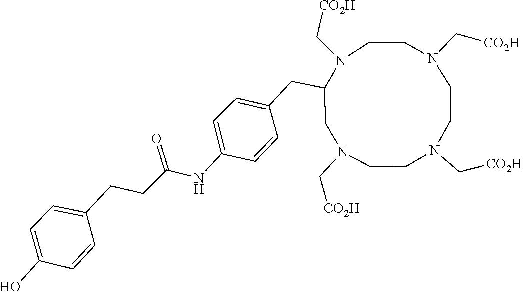 [SMILES: O=C(O)CN1CCN(CC(=O)O)CCN(CC(=O)O)C(Cc2ccc(NC(=O)CCc3ccc(O)cc3)cc2)CN(CC(=O)O)CC1] | (3) |

### Salts

In another aspect, disclosed are salts of the compound of Formula (I). In certain embodiments, the salt comprises a metal counterion, hi certain embodiments, the metal counterion is bismuth, lead, yttrium, cadmium, mercury, actinium, thorium, strontium, or a lanthanide. In certain embodiments, the metal counterion has an oxidation state of +3. In certain embodiments, the metal counterion is yttrium, praseodymium, or lutetium. In certain embodiments, the metal counterion is yttrium. In certain embodiments, the metal counterion is Y³⁺. In certain embodiments, the metal counterion is praseodymium. In certain embodiments, the metal counterion is P³⁺.

### Chelate Complex

In another aspect, disclosed are chelate complexes comprising the compound of Formula (I) or a salt thereof. A chelate complex is a chemical compound composed of a metal ion and a chelating agent. A chelating agent forms one or more bonds to a single metal ion. In the present disclosure, the compound of Formula (I) may be a chelating agent. In certain embodiments, the chelate complex comprising a compound of Formula (I), or a salt thereof, is a monovalent chelate complex. In certain embodiments, the chelate complex comprising a compound of Formula (I), or a salt thereof, is a divalent chelate complex. In certain embodiments, the chelate complex comprising a compound of Formula (I), or a salt thereof, is a trivalent chelate complex.

In certain embodiments, the metal ion is bismuth, lead, yttrium, cadmium, mercury, actinium, thorium, strontium, or a lanthanide, hi certain embodiments, the metal ion is yttrium, praseodymium, or lutetium. In certain embodiments, the metal ion has an oxidation state of +3. In certain embodiments, the metal ion is yttrium. In certain embodiments, the metal ion is Y³⁺. In certain embodiments, the metal ion is praseodymium. In certain embodiments, the metal ion is P³⁺.

In certain embodiments, the chelate complex comprising the compound of Formula (I), or a salt thereof, is any of the formula of Table 2.

TABLE 2

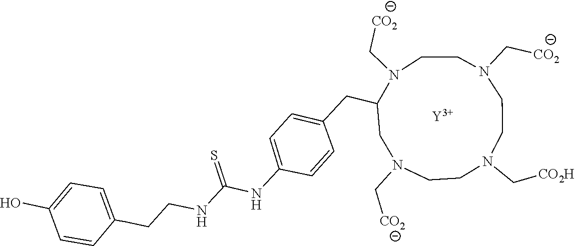

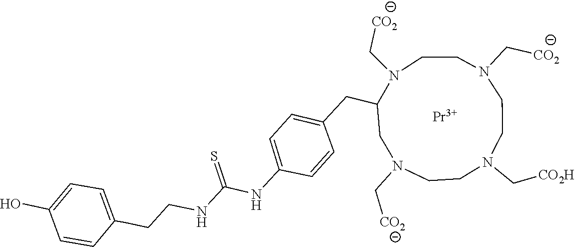

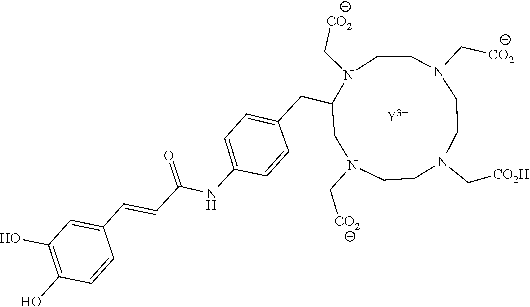

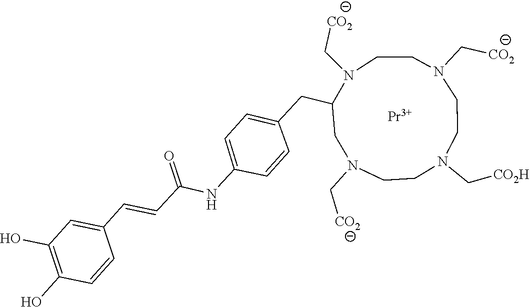

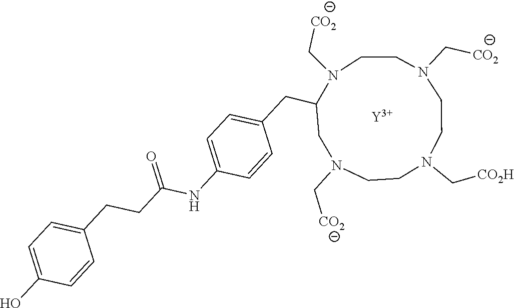

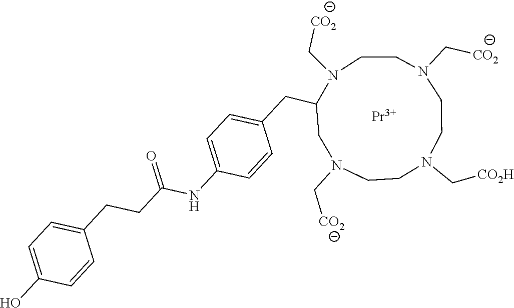

### Kits

Also encompassed by the disclosure are kits. The kits provided may comprise a compound, salt, or chelate complex described herein and a container (e.g., a vial, ampule, bottle, syringe, and/or dispenser package, or other suitable container). In certain embodiments, the kit further comprises an activating enzyme. In certain embodiments, the enzyme is a peroxidase. In certain embodiments, the enzyme is horseradish peroxidase.

Thus, in one aspect, provided are kits including a container comprising a compound, salt, or chelate complex described herein. In certain embodiments, the kits are useful for assaying methods to detect an analyte (e.g., a protein from human tissue, nucleic acids). In certain embodiments, a kit described herein further includes instructions for using the kit.

### Methods of Use

Provided herein are DOTA-containing compounds (e.g., compounds of Formula (I)) and complexes that are reactive with an enzyme activator (e.g., a peroxidase) and covalently bind a target analyte (e.g., a protein, nucleic acids) in a catalyzed reporter deposition assaying method. The macrocyclic DOTA portion of the molecule may then act as a reporter group which can be detected directly (e.g., mass spectrometry based imaging) or indirectly (e.g., DOTA antibody coupled to chromogenic deposition of a light-emitting compound and visualization with light or fluorescence microscopy). The compounds are most useful in the methods and uses described herein when they comprise a chelate complex having a metal counterion (e.g., lanthanides, yttrium, praesodynium). The DOTA-containing compounds and complexes are advantageous over existing reporter compounds and complexes because the DOTA moiety allows detection of the reporter moiety by use of mass spectrometry imaging microscopy.

In one aspect, disclosed is a method of detecting an analyte, the method comprising: reacting an enzyme with a chelate complex (e.g., comprising a compound of Formula (I)) described herein to form an oxidized chelate complex; contacting the oxidized chelate complex with a biological sample; and detecting the analyte in the biological sample.

In certain embodiments, the method further comprises covalent binding of the oxidized chelate complex with the analyte.

In certain embodiments, the biological sample is immobilized.

In certain embodiments, the analyte comprises a protein derived from human tissue.

In certain embodiments, the enzyme is a peroxidase. In certain embodiments, the enzyme is horseradish peroxidase. In certain embodiments, the analyte does not react with the enzyme.

In certain embodiments, the detecting of the analyte is direct or indirect. In certain embodiments, the detecting of the analyte is indirect. In certain embodiments, the detecting of the analyte comprises adding a DOTA chelate-specific antibody coupled to chromogenic deposition of a light-emitting compound (e.g., 3,3′-diaminobenzidine tetrahydrochloride) and visualization with light or fluorescence microscopy.

In certain embodiments, the detecting of the analyte is direct. In certain embodiments, the detecting of the analyte comprises mass spectrometry imaging based microscopy. In certain embodiments, the detecting is achieved by employing mass spectrometry imaging based microscopy. In certain embodiments, the detecting is achieved by employing multiplexed ion beam imaging (MIBI). In certain embodiments, the detecting is achieved by employing Imaging Mass Cytometry™ (IMC™).

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

Compounds of Formula I may be prepared using the synthetic schemes described below.

### Preparation of Exemplary Compounds

Compound 1 was prepared by the following synthetic scheme.

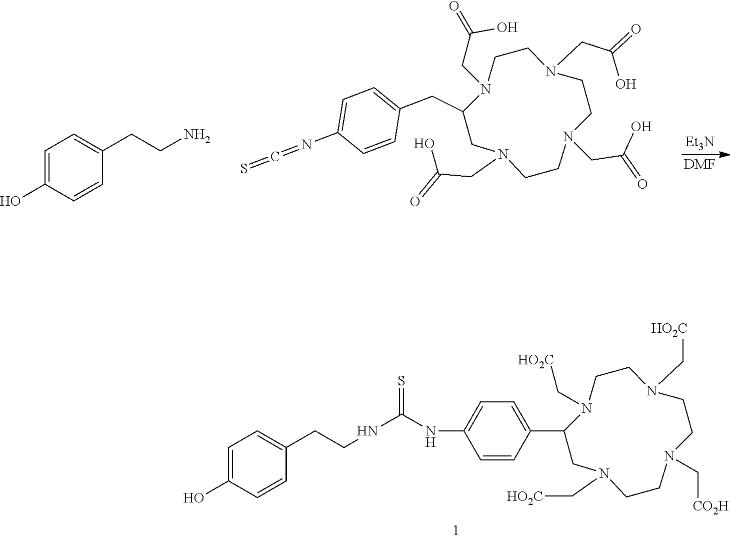

Compound 2 was prepared by the following synthetic scheme.

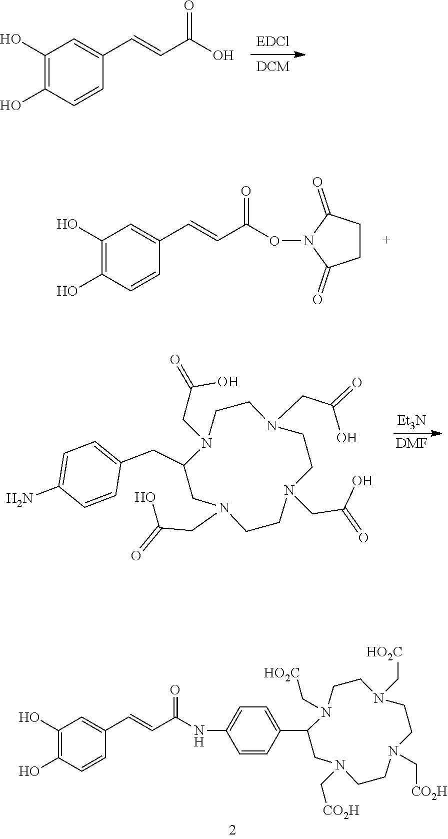

Compound 3 was prepared by the following synthetic scheme.

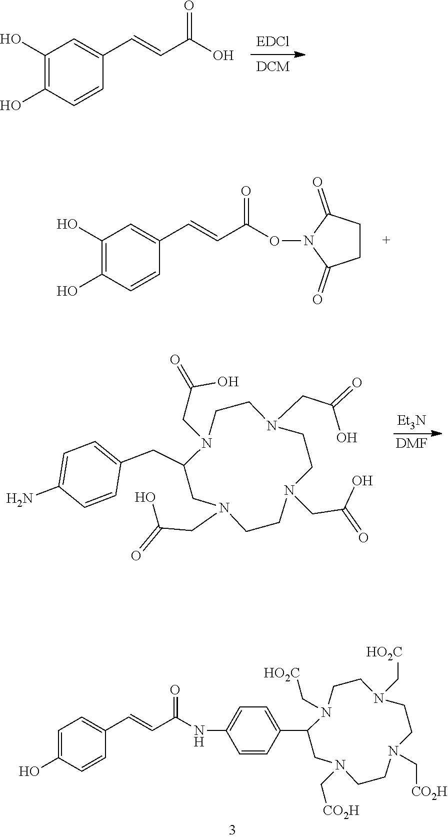

Metal loading of the exemplary compounds was accomplished by the following scheme.

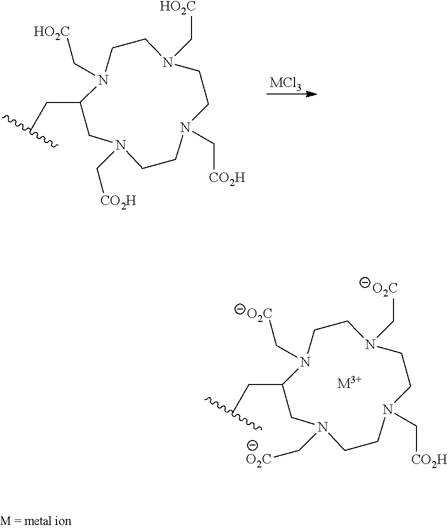

### Assays

Assays were performed to demonstrate the ability of chelate complexes of the exemplary compounds to function in a catalyzed reporter deposition assaying method.

Tables A-F below describe the stepwise procedure of the assays.

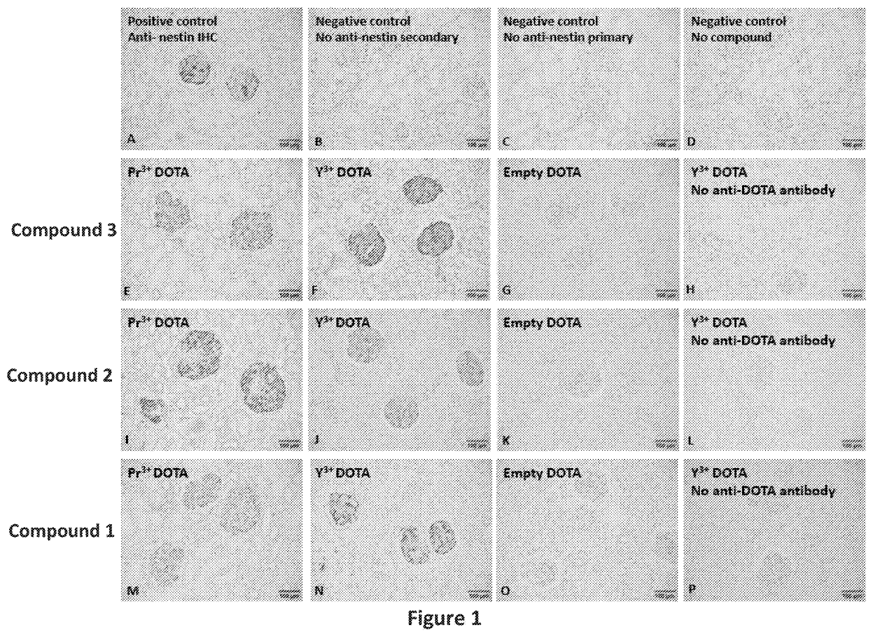

Table A (anti-nestin positive control) provides the assay procedure for demonstrating the expression of the intermediate filament protein nestin detected in normal human kidney podocytes using a goat anti-nestin polyclonal antibody with a rabbit anti-goat secondary horseradish peroxidase/3,3′-diaminobenzidine (DAB) chromogenic assay and standard light microscopy (FIG. 1A).

Tables B and C provide the assay procedures for demonstrating that the chromogenic signal for nestin expression is not seen with negative control conditions including without anti-nestin primary antibody (Table C; FIG. 1C) or without secondary antibody (Table B; FIG. 1B).

Table D provides the assay procedure for demonstrating that the nestin-specific signal is not seen without the DOTA chelate complex in the anti-DOTA antibody assay (FIG. 1D).

Table E provides the assay procedure for evaluating exemplary compounds 1, 2, and 3 having DOTA complexed to Pr³⁺ (FIG. 1E, FIG. 1I, FIG. 1M) or Y³⁺ (FIG. 1F, FIG. 1J, FIG. 1N) or not complexed (FIG. 1G, FIG. 1K, FIG. 1O). In each assay the complex was oxidized by peroxidase and deposited at the site of the anti-nestin antibody. This specific deposition of complex was detected with an anti-DOTA chelate antibody (clone 2D12.5) chromogenic assay using DAB. Empty DOTA is not recognized by the anti-DOTA chelate antibody (clone 2D12.5), therefore chromogenic signal was not seen in these assay conditions (FIG. 1G, FIG. 1K, FIG. 1O).

Table F provides the assay procedure for evaluating exemplary compounds 1, 2, and 3 having DOTA complexed to Y³⁺ (FIG. 1H, FIG. 1L, FIG. 1P) but without addition of the anti-DOTA antibody to the assay. Nestin-specific signal was not seen without anti-DOTA chelate antibody (FIG. 1H, FIG. 1L, FIG. 1P).

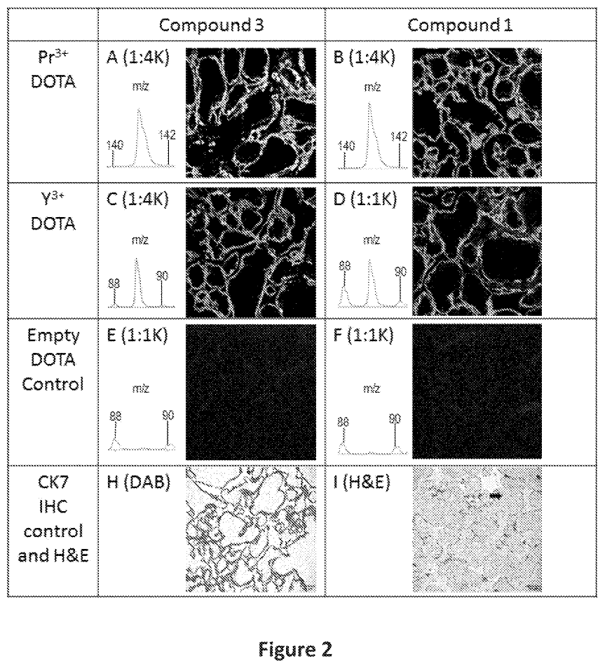

FIG. 2 confirms that the disclosed compounds can be detected by a mass spectrometry imaging device in an assay. Images were generated using a MIBIscope I multiplexed ion beam imaging device from IONpath (Menlo Park, Calif.). The parenthetical terms 1:4K, 1:2K, and 1:1K refer to the dilution of the compound used in the assay. In particular, compounds 1 and 3 complexed to Pr³⁺ (FIGS. 2A, 2C) or Y³⁺ (FIGS. 2B, 2D) were detected directly in human placenta samples using the mass spectrometry imaging device.

TABLE A

| | | | |
|---|---|---|---|
| Step Reagent | Inc. (min): | Temperature: | Supplier Santo Cruze |
| 1 Goat anti- | 30:00 | Ambient | Cat# SC-21248; |
| human | | | Lot# A2313 |
| NESTIN | | | Dispense type: 150 μL |
| Step type: Reagent | | | |
| Step Reagent | Inc. (min): | Temperature: | Supplier: Leica |
| 2 *BondWashSolution | 0:00 | Ambient | Microsystems |
| Step type: Wash | | | Dispense type. 150 μL |
| Step Reagent | Inc. (min): | Temperature: | Supplier: Leica |
| 3 *BondWashSolution | 0:00 | Ambient | Microsystems |
| Step type: Wash | | | Dispense type. 150 μL |
| Step Reagent | Inc. (min): | Temperature: | Supplier: Leica |
| 4 *BondWashSolution | 0:00 | Ambient | Microsystems |
| Step type: Wash | | | Dispense type. 150 μL |
| Step Reagent | Inc. (min): | Temperature: | Supplier: Leica |
| 5 *BondWashSolution | 2:00 | Ambient | Microsystems |
| Step type: Wash | | | Dispense type. 150 μL |
| Step Reagent | Inc. (min): | Temperature: | Supplier: Leica |
| 6 *BondWashSolution | 2:00 | Ambient | Microsystems |
| Step type: Wash | | | Dispense type. 150 μL |
| Step Reagent | Inc. (min): | Temperature: | Supplier: Leica |
| 7 *BondWashSolution | 2:00 | Ambient | Microsystems |
| Step type: Wash | | | Dispense type. 150 μL |
| Step Reagent | Inc. (min): | Temperature: | Supplier: Vector Lab |
| 8 Rabbit anti- | 8:00 | Ambient | Cat# BA-5000 |
| goat IgG | | | Dispense type: 150 μL |
| Step type: Reagent | | | |
| Step Reagent | Inc. (min): | Temperature: | Supplier: Leica |
| 9 *BondWashSolution | 2:00 | Ambient | Microsystems |
| Step type: Wash | | | Dispense type: 150 μL |
| Step Reagent | Inc. (min): | Temperature: | Supplier: Leica |
| 10 *BondWashSolution | 2:00 | Ambient | Microsystems |
| Step type: Wash | | | Dispense type: 150 μL |
| Step Reagent | Inc. (min): | Temperature: | Supplier: Leica |
| 11 *BondWashSolution | 2:00 | Ambient | Microsystems |
| Step type: Wash | | | Dispense type: 150 μL |
| Step Reagent | Inc. (min): | Temperature: | Supplier: Leica |
| 12 *Polymer | 8:00 | Ambient | Microsystems |
| Step type: Reagent | | | Dispense type: 150 μL |
| Step Reagent | Inc. (min): | Temperature: | Supplier: Leica |
| 13 *BondWashSolution | 2:00 | Ambient | Microsystems |
| Step type: Wash | | | Dispense type: 150 μL |
| Step Reagent | Inc. (min): | Temperature: | Supplier: Leica |
| 14 *BondWashSolution | 2:00 | Ambient | Microsystems |
| Step type: Wash | | | Dispense type: 150 μL |
| Step Reagent | Inc. (min): | Temperature: | Supplier: Leica |
| 15 *BondWashSolution | 2:00 | Ambient | Microsystems |
| Step type: Wash | | | Dispense type: 150 μL |
| Step Reagent | Inc. (min): | Temperature: | Supplier: Leica |
| 16 *BondWashSolution | 0:00 | Ambient | Microsystems |
| Step type: Wash | | | Dispense type: 150 μL |
| Step Reagent | Inc. (min): | Temperature: | Supplier: Leica |
| 17 *Peroxide Block | 5:00 | Ambient | Microsystems |
| Step type: Reagent | | | Dispense type: 150 μL |
| Step Reagent | Inc. (min): | Temperature: | Supplier: Leica |
| 18 *BondWashSolution | 0:00 | Ambient | Microsystems |
| Step type: Wash | | | Dispense type: 150 μL |
| Step Reagent | Inc. (min): | Temperature: | Supplier: Leica |
| 19 *BondWashSolution | 0.00 | Ambient | Microsystems |
| Step type: Wash | | | Dispense type. Open |
| Step Reagent | Inc. (min): | Temperature: | Supplier: Leica |
| 20 *BondWashSolution | 0:00 | Ambient | Microsystems |
| Step type: Wash | | | Dispense type: 150 μL |
| Step Reagent | Inc. (min): | Temperature: | Supplier: Not applicable |
| 21 *Deionized Water | 0:00 | Ambient | Dispense type: 150 μL |
| Step type: Reagent | | | |
| Step Reagent | Inc. (min): | Temperature: | Supplier: Not applicable |
| 22 *Delonized Water | 0:00 | Ambient | Dispense type: 150 μL |
| Step type: Wash | | | |
| Step Reagent | Inc. (min): | Temperature: | Supplier: Leica |
| 23 *MixedDABRefine | 2:00 | Ambient | Microsystems |
| Step type: Reagent | | | Dispense type: 150 μL |
| Step Reagent | Inc. (min): | Temperature: | Supplier: Leica |
| 24 *MixedDABRefine | 5:00 | Ambient | Microsystems |
| Step type: Reagent | | | Dispense type: 150 μL |
| Step Reagent | Inc. (min): | Temperature: | Supplier: Not applicable |
| 25 *Deionized Water | 0:00 | Ambient | Dispense type: 150 μL |
| Step type: Wash | | | |
| Step Reagent | Inc. (min): | Temperature: | Supplier: Not applicable |
| 26 *Deionized Water | 0:00 | Ambient | Dispense type: 150 μL |
| Step type: Wash | | | |
| Step Reagent | Inc. (min): | Temperature: | Supplier: Not applicable |
| 27 *Deionized Water | 0:00 | Ambient | Dispense type: 150 μL |
| Step type: Wash | | | |
| Step Reagent | Inc. (min): | Temperature: | Supplier: Leica |
| 28 *Hematoxylin | 10:00 | Ambient | Microsystems |
| Step type: Reagent | | | Dispense type: 150 μL |
| Step Reagent | Inc. (min): | Temperature: | Supplier: Not applicable |
| 29 *Deionized Water | 0:00 | Ambient | Dispense type: 150 μL |
| Step type: Wash | | | |
| Step Reagent | Inc. (min): | Temperature: | Supplier: Leica |
| 30 *BondWashSolution | 1:00 | Ambient | Microsystems |
| Step type: Wash | | | Dispense type: 150 μL |
| Step Reagent | Inc. (min): | Temperature: | Supplier: Leica |
| 31 *BontWashSolution | 0:00 | Ambient | Microsystems |
| Step type: Wash | | | Dispense type: 150 μL |
| Step Reagent | Inc. (min): | Temperature: | Supplier: Not applicable |
| 32 *Deionized Water | 0:00 | Ambient | Dispense type: 150 μL |
| Step type: Wash | | | |

TABLE B

| | | | |
|---|---|---|---|
| Step Reagent | Inc. (min): 30:00 | Temperature: Ambient | Supplier: Santa Cruze |
| 1 Goat anti- | | | Cat# SC-21248; |
| human | | | Lot# A2313 |
| NESTIN | | | Dispense type: 150 μL |
| Step type: Reagent | | | |
| Step Reagent | Inc. (min): 0:00 | Temperature: Ambient | Supplier: Leica Microsystems |
| 2 *BondWashSolution | | | Dispense type: 150 μL |
| Step type: Wash | | | |
| Step Reagent | Inc. (min): 0:00 | Temperature: Ambient | Supplier: Leica Microsystems |
| 3 *BondWashSolution | | | Dispense type: 150 μL |
| Step type: Wash | | | |
| Step Reagent | Inc. (min): 0:00 | Temperature: Ambient | Supplier: Leica Microsystems |
| 4 *BondWashSolution | | | Dispense type: 150 μL |
| Step type: Wash | | | |
| Step Reagent | Inc. (min): 2:00 | Temperature: Ambient | Supplier: Leica Microsystems |
| 5 *BondWashSolution | | | Dispense type: 150 μL |
| Step type: Wash | | | |
| Step Reagent | Inc. (min): 2:00 | Temperature: Ambient | Supplier: Leica Microsystems |
| 6 *BondWashSolution | | | Dispense type: 150 μL |
| Step type: Wash | | | |
| Step Reagent | Inc. (min): 2:00 | Temperature: Ambient | Supplier: Leica Microsystems |
| 7 *BondWashSolution | | | Dispense type: 150 μL |
| Step type: Wash | | | |
| Step Reagent | Inc. (min): 8:00 | Temperature: Ambient | Supplier: VWR |
| 8 TBS-t | | | Cat# J640-1L |
| Step type: Reagent | | | Dispense type: 150 μL |
| Step Reagent | Inc. (min): 2.00 | Temperature: Ambient | Supplier: Leica Microsystems |
| 9 *BondWashSolution | | | Dispense type: 150 μL |
| Step type: Wash | | | |
| Step Reagent | Inc. (min): 2:00 | Temperature: Ambient | Supplier: Leica Microsystems |
| 10 *BondWashSolution | | | Dispense type: 150 μL |
| Step type: Wash | | | |
| Step Reagent | Inc. (min ): 2:00 | Temperature: Ambient | Supplier: Leica Microsystems |
| 11 *BondWashSolution | | | Dispense type: 150 μL |
| Step type: Wash | | | |
| Step Reagent | Inc. (min): 8:00 | Temperature: Ambient | Supplier: Leica Microsystems |
| 12 *Polymer | | | Dispense type: 150 μL |
| Step type: Reagent | | | |
| Step Reagent | Inc. (min): 2:00 | Temperature: Ambient | Supplier: Leica Microsystems |
| 13 *BondWashSolution | | | Dispense type: 150 μL |
| Step type: Wash | | | |
| Step Reagent | Inc. (min): 2.00 | Temperature: Ambient | Supplier: Leica Microsystems |
| 14 *BondWashSolution | | | Dispense type: 150 μL |
| Step type: Wash | | | |
| Step Reagent | Inc. (min): 2:00 | Temperature: Ambient | Supplier: Leica Microsystems |
| 15 *BondWashSolution | | | Dispense type: 150 μL |
| Step type: Wash | | | |
| Step Reagent | Inc. (min): 0:00 | Temperature: Ambient | Supplier: Leica Microsystems |
| 16 *BondWashSolution | | | Dispense type: 150 μL |
| Step type: Wash | | | |
| Step Reagent | Inc. (min): 5:00 | Temperature: Ambient | Supplier: Leica Microsystems |
| 17 *Peroxide Block | | | Dispense type: 150 μL |
| Step type: Reagent | | | |
| Step Reagent | Inc. (min): 0:00 | Temperature: Ambient | Supplier: Leica Microsystems |
| 18 *BondWashSolution | | | Dispense type: 150 μL |
| Step type: Wash | | | |
| Step Reagent | Inc. (min): 0.00 | Temperature: Ambient | Supplier: Leica Microsystems |
| 19 *BondWashSolution | | | Dispense type: Open |
| Step type: Wash | | | |
| Step Reagent | Inc. (min): 0:00 | Temperature: Ambient | Supplier: Leica Microsystems |
| 20 *BondWashSolution | | | Dispense type: 150 μL |
| Step type: Wash | | | |
| Step Reagent | Inc. (min): 0.00 | Temperature: Ambient | Supplier: Notapplicable |
| 21 *Deionized Water | | | Dispense type: 150 μL |
| Step type: Reagent | | | |
| Step Reagent | Inc. (min): 0:00 | Temperature: Ambient | Supplier: Notapplicable |
| 22 *Deionized Water | | | Dispense type: 150 μL |
| Step type: Wash | | | |
| Step Reagent | Inc. (min): 2:00 | Temperature: Ambient | Supplier: Leica Microsystems |
| 23 *MixedDABRefine | | | Dispense type: 150 μL |
| Step type: Reagent | | | |
| Step Reagent | IBC. (min): 5:00 | Temperature: Ambient | Supplier: Leica Microsystems |
| 24 *MixedDABRefine | | | Dispense type: 150 μL |
| Step type: Reagent | | | |
| Step Reagent | Inc. (min): 0:00 | Temperature: Ambient | Supplier: Notapplicable |
| 25 *Deionized Water | | | Dispense type: 150 μL |
| Step type: Wash | | | |
| Step Reagent | Inc. (min): 0:00 | Temperature: Ambient | Supplier: Notapplicable |
| 26 *Deionized Water | | | Dispense type: 150 μL |
| Step type: Wash | | | |
| Step Reagent | Inc. (min): 0:00 | Temperature: Ambient | Supplier: Notapplicable |
| 27 *Deionized Water | | | Dispense type: 150 μL |
| Step type: Wash | | | |
| Step Reagent | Inc. (min): 10:00 | Temperature: Ambient | Supplier: Leica Microsystems |
| 28 *Hematoxylin | | | Dispense type: 150 μL |
| Step type: Reagent | | | |
| Step Reagent | Inc. (min): 0:00 | Temperature: Ambient | Supplier: Leica Microsystems |
| 29 *Deionized Water | | | Dispense type: 150 μL |
| Step type: Wash | | | |
| Step Reagent | Inc. (min): 1:00 | Temperature: Ambient | Supplier: Leica Microsystems |
| 30 *BondWashSolution | | | Dispense type: 150 μL |
| Step type: Wash | | | |
| Step Reagent | Inc. (min): 0:00 | Temperature: Ambient | Supplier: Leica Microsystems |
| 31 *BondWashSolution | | | Dispense type: 150 μL |
| Step type: Wash | | | |
| Step Reagent | Inc. (min): 0:00 | Temperature: Ambient | Supplier: Notapplicable |
| 32 *Deioniz ed Water | | | Dispense type: 150 μL |
| Step type: Wash | | | |

TABLE C

| | | | |
|---|---|---|---|
| Step Reagent | Inc. (min): 30:00 | Temperature: Ambient | Supplier: VWR |
| 1 TBS-t | | | Cat# J640-1L |
| Step type: Reagent | | | Dispense type: 150 μL |
| Step Reagent | Inc. (min): 0:00 | Temperature: Ambient | Supplier: Leica Microsystems |
| 2 *BondWashSolution | | | Dispense type: 150 μL |
| Step type: Wash | | | |
| Step Reagent | Inc. (min): 0:00 | Temperature: Ambient | Supplier: Leica Microsystems |
| 3 *BondWashSolution | | | Dispense type: 150 μL |
| Step type: Wash | | | |
| Step Reagent | Inc. (min): 0:00 | Temperature: Ambient | Supplier: Leica Microsystems |
| 4 *BondWashSolution | | | Dispense type: 150 μL |
| Step type: Wash | | | |
| Step Reagent | Inc. (min): 2:00 | Temperature: Ambient | Supplier: Leica Microsystems |
| 5 *BondWashSolution | | | Dispense type: 150 μL |
| Step type: Wash | | | |
| Step Reagent | Inc. (min): 2:00 | Temperature: Ambient | Supplier: Leica Microsystems |
| 6 *BondWashSolution | | | Dispense type: 150 μL |
| Step type: Wash | | | |
| Step Reagent | Inc. (min): 2:00 | Temperature: Ambient | Supplier: Leica Microsystems |
| 7 *BondWashSolution | | | Dispense type: 150 μL |
| Step type: Wash | | | |
| Step Reagent | Inc. (min): 8:00 | Temperature: Ambient | Supplier: Vector Lab |
| 8 Rabbit anti- | | | Cat# BA-5000 |
| goat IgG | | | Dispense type: 150 μL |
| Step type: Reagent | | | |
| Step Reagent | Inc. (min): 2:00 | Temperature: Ambient | Supplier: Leica Microsystems |
| 9 *BondWashSolution | | | Dispense type: 150 μL |
| Step type: Wash | | | |
| Step Reagent | Inc. (min): 2:00 | Temperature: Ambient | Supplier: Leica Microsystems |
| 10 *BondWashSolution | | | Dispense type: 150 μL |
| Step type: Wash | | | |
| Step Reagent | Inc. (min): 2:00 | Temperature: Ambient | Supplier: Leica Microsystems |
| 11 *BondWashSolution | | | Dispense type: 150 μL |
| Step type: Wash | | | |
| Step Reagent | Inc. (min): 8:00 | Temperature: Ambient | Supplier: Leica Microsystems |
| 12 *Polymer | | | Dispense type: 150 μL |
| Step type: Reagent | | | |
| Step Reagent | Inc. (min): 2:00 | Temperature: Ambient | Supplier: Leica Microsystems |
| 13 *BondWashSolution | | | Dispense type: 150 μL |
| Step type: Wash | | | |
| Step Reagent | Inc. (min): 2:00 | Temperature: Ambient | Supplier: Leica Microsystems |
| 14 *BondWashSolution | | | Dispense type: 150 μL |
| Step type: Wash | | | |
| Step Reagent | Inc. (min): 2:00 | Temperature: Ambient | Supplier: Leica Microsystems |
| 15 *BondWashSolution | | | Dispense type: 150 μL |
| Step type: Wash | | | |
| Step Reagent | Inc. (min): 0:00 | Temperature: Ambient | Supplier: Leica Microsystems |
| 16 *BondWashSolution | | | Dispense type: 150 μL |
| Step type: Wash | | | |
| Step Reagent | Inc. (min): 5:00 | Temperature: Ambient | Supplier: Leica Microsystems |
| 17 *Peroxide Block | | | Dispense type: 150 μL |
| Step type: Reagent | | | |
| Step Reagent | Inc. (min): 0:00 | Temperature: Ambient | Supplier: Leica Microsystems |
| 18 *BondWashSolution | | | Dispense type: 150 μL |
| Step type: Wash | | | |
| Step Reagent | Inc. (min): 0:00 | Temperature: Ambient | Supplier: Leica Microsystems |
| 19 *BondWashSolution | | | Dispense type: Open |
| Step type: Wash | | | |
| Step Reagent | Inc. (min): 0:00 | Temperature: Ambient | Supplier: Leica Microsystems |
| 20 *BondWashSolution | | | Dispense type: 150 μL |
| Step type: Wash | | | |
| Step Reagent | Inc. (min): 0:00 | Temperature: Ambient | Supplier: Notapplicable |
| 21 *Deionized Water | | | Dispense type: 150 μL |
| Step type: Reagent | | | |
| Step Reagent | Inc. (min): 0:00 | Temperature: Ambient | Supplier: Notapplicable |
| 22 *Deionized Water | | | Dispense type: 150 μL |
| Step type: Wash | | | |
| Step Reagent | Inc. (min): 2:00 | Temperature: Ambient | Supplier: Leica Microsystems |
| 23 *MixedDABRefine | | | Dispense type: 150 μL |
| Step type: Reagent | | | |
| Step Reagent | Inc. (min): 5:00 | Temperature: Ambient | Supplier: Leica Microsystems |
| 24 *MixedDABRefine | | | Dispense type: 150 μL |
| Step type: Reagent | | | |
| Step Reagent | Inc. (min): 0:00 | Temperature: Ambient | Supplier: Notapplicable |
| 25 *Deionized Water | | | Dispense type: 150 μL |
| Step type: Wash | | | |
| Step Reagent | Inc. (min): 0:00 | Temperature: Ambient | Supplier: Notapplicable |
| 26 *Deionized Water | | | Dispense type: 150 μL |
| Step type: Wash | | | |
| Step Reagent | Inc. (min): 0:00 | Temperature: Ambient | Supplier: Notapplicable |
| 27 *Deionized Water | | | Dispense type: 150 μL |
| Step type: Wash | | | |
| Step Reagent | Inc. (min): 10:00 | Temperature: Ambient | Supplier: Leica Microsystems |
| 28 *Hematoxylin | | | Dispense type: 150 μL |
| Step type: Reagent | | | |
| Step Reagent | Inc. (min): 0:00 | Temperature: Ambient | Supplier: Notapplicable |
| 29 *Deionized Water | | | Dispense type: 150 μL |
| Step type: Wash | | | |
| Step Reagent | Inc. (min): 1:00 | Temperature: Ambient | Supplier: Leica Microsystems |
| 30 *BondWashSolution | | | Dispense type: 150 μL |
| Step type: Wash | | | |
| Step Reagent | Inc. (min): 0:00 | Temperature: Ambient | Supplier: Leica Microsystems |
| 31 *BondWashSolution | | | Dispense type: 150 μL |
| Step type: Wash | | | |
| Step Reagent | Inc. (min): 0:00 | Temperature: Ambient | Supplier: Notapplicable |
| 32 *Deionized Water | | | Dispense type: 150 μL |
| Step type: Wash | | | |

TABLE D

| | | | |
|---|---|---|---|
| Step Reagent | Inc. (min): 30:00 | Temperature: Ambient | Supplier: SantaCruze, (Cat# SC-21248; |
| 1 Goat anti- | | | Lot# A2313) |
| human | | | Dispense type: 150 μL |
| NESTIN | | | |
| Step type: Reagent | | | |
| Step Reagent | Inc. (min): 2:00 | Temperature: Ambient | Supplier: Leica Microsystems |
| 2 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 3 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 4 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 5 *BondWashSolution | | | Cat# AR9590 |
| Step type: Wash | | | Dispense type: 150 μL |
| Step Reagent | Inc. (min): 8:00 | Temperature: Ambient | Supplier: Vector Lab (Cat# BA-5000) |
| 6 Rabbit anti- | | | Dispense type: 150 μL |
| goat | | | |
| secondary | | | |
| antibody | | | |
| Step type: Reagent | | | |
| Step Reagent | Inc. (min): 2:00 | Temperature: Ambient | Supplier: Leica Microsystems |
| 7 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 8 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 9 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 10 *BondWashSolution | | | Cat# AR9590 |
| Step type: Wash | | | Dispense type: 150 μL |
| Step Reagent | Inc. (min): 8:00 | Temperature: Ambient | Supplier: Bond Polymer Refine Detection kit |
| 11 *Polymer | | | Cat#: DS9800 |
| Step type: Reagent | | | Dispense type: 150 μL |
| Step Reagent | Inc. (min): 2:00 | Temperature: Ambient | Supplier: Leica Microsystems |
| 12 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 13 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 14 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 15 *BondWashSolution | | | Cat# AR9590 |
| Step type: Wash | | | Dispense type: 150 μL |
| Step Reagent | Inc. (min): 10:00 | Temperature: Ambient | Supplier: VWR |
| 16 TBS-t | | | Cat# J640-1L |
| Step type: Reagent | | | Dispense type: 150 μL |
| Step Reagent | Inc. (min): 2:00 | Temperature: Ambient | Supplier: Leica Microsystems |
| 17 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 18 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 19 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 20 *BondWashSolution | | | Cat# AR9590 |
| Step type: Wash | | | Dispense type: 150 μL |
| Step Reagent | Inc. (min): 0:00 | Temperature: 100° C. | Supplier: Perkin Elmer |
| 21 AR9 Buffer | | | Cat#AR900250ML |
| Step type: Reagent | | | Dispense type: 150 μL |
| Step Reagent | Inc. (min): 20:00 | Temperature: 100° C. | Supplier: Perkin Elmer |
| 22 AR9 Buffer | | | Cat#AR900250ML |
| Step type: Reagent | | | Dispense type: 150 μL |
| Step Reagent | Inc. (min): 0:00 | Temperature: 100 | Supplier: Perkin Elmer |
| 23 AR9 Buffer | | | Cat#AR900250ML |
| Step type: Reagent | | | Dispense type: 150 μL |
| Step Reagent | Inc. (min): 2:00 | Temperature: Ambient | Supplier: Perkin Elmer |
| 24 AR9 Buffer | | | Cat#AR900250ML |
| Step type: Reagent | | | Dispense type: 150 μL |
| Step Reagent | Inc. (min): 0:00 | Temperature: Ambient | Supplier: Perkin Elmer |
| 25 AR9 Buffer | | | Cat#AR900250ML |
| Step type: Reagent | | | Dispense type: 150 μL |
| Step Reagent | Inc. (min): 0:00 | Temperature: Ambient | Supplier: Leica Microsystems |
| 26 *BondWashSolution | | | Cat# AR9590 |
| Step type: Wash | | | Dispense type: 150 μL |
| Step Reagent | Inc. (min): 10:00 | Temperature: Ambient | Supplier: Vector Lab |
| 27 Normal horse serum | | | Cat# MP-7402 kit |
| Step type: Reagent | | | Dispense type: 150 μL |
| Step Reagent | Inc. (min): 30:00 | Temperature: Ambient | Supplier: Creative BioLabs (2D12.5) |
| 28 Mouse | | | Cat# PABW-133 |
| anti- | | | Dispense type: 150 μL |
| DOTA | | | |
| antibody | | | |
| 2D12.5 | | | |
| Step type: Reagent | | | |
| Step Reagent | Inc. (min): 2:00 | Temperature: Ambient | Supplier: Leica Microsystems |
| 29 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 30 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 31 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 32 *BondWashSolution | | | Cat# AR9590 |
| Step type: Wash | | | Dispense type: 150 μL |
| Step Reagent | Inc. (min): 20:00 | Temperature: Ambient | Supplier: Vector Lab |
| 33 Horse anti-mouse | | | Cat# MP-7402 kit |
| IgG | | | Dispense type: 150 μL |
| Step type: Reagent | | | |
| Step Reagent | Inc. (min): 2:00 | Temperature: Ambient | Supplier: Leica Microsystems |
| 34 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 35 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 36 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 37 *BondWashSolution | | | Cat# AR9590 |
| Step type: Wash | | | Dispense type: 150 μL |
| Step Reagent | Inc. (min): 8:00 | Temperature: Ambient | Supplier: Bond Polymer Refine Detection kit |
| 38 *Peroxide Block | | | Cat# DS9800 |
| Step type: Reagent | | | Dispense type: 150 μL |
| Step Reagent | Inc. (min): 0:00 | Temperature: Ambient | Supplier: Leica Microsystems |
| 39 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 40 *BondWashSolution | | | Cat# AR9590 |
| Step type: Wash | | | Dispense type: Open |
| Step Reagent | Inc. (min): 0:00 | Temperature: Ambient | Supplier: Leica Microsystems |
| 41 *BondWashSolution | | | Cat# AR9590 |
| Step type: Wash | | | Dispense type: 150 μL |
| Step Reagent | Inc. (min): 0:00 | Temperature: Ambient | Supplier: B. BRAUN Medical Inc. #R5000-01 |
| 42 *Deionized Water | | | Dispense type: 150 μL |
| Step type: Reagent | | | |
| Step Reagent | Inc. (min): 0:00 | Temperature: Ambient | Supplier: B. BRAUN Medical Inc. #R5000-01 |
| 43 *Deionized Water | | | Dispense type: 150 μL |
| Step type: Wash | | | |
| Step Reagent | Inc. (min): 2:00 | Temperature: Ambient | Supplier: Bond Polymer Refine Detection kit |
| 44 *MixedDABRefine | | | Cat# DS9800 |
| Step type: Reagent | | | Dispense type: 150 μL |
| Step Reagent | Inc. (min): 5:00 | Temperature: Ambient | Supplier: Bond Polymer Refine Detection kit |
| 45 *MixedDABRefine | | | Cat# DS9800 |
| Step type: Reagent | | | Dispense type: 150 μL |
| Step Reagent | Inc. (min): 0:00 | Temperature: Ambient | Supplier: B. BRAUN Medical Inc. #R5000-01 |
| 46 *Deionized Water | | | Dispense type: 150 μL |
| Step type: Wash | | | |
| Step Reagent | Inc. (min): 0:00 | Temperature: Ambient | Supplier: B. BRAUN Medical Inc. #R5000-01 |
| 47 *Deionized Water | | | Dispense type: 150 μL |
| Step type: Wash | | | |
| Step Reagent | Inc. (min): 0:00 | Temperature: Ambient | Supplier: B. BRAUN Medical Inc. #R5000-01 |
| 48 *Deionized Water | | | Dispense type: 150 μL |
| Step type: Wash | | | |
| Step Reagent | Inc. (min): 10:00 | Temperature: Ambient | Supplier: Bond Polymer Refine Detection kit |
| 49 *Hematoxylin | | | Cat# DS9800 |
| Step type: Reagent | | | Dispense type: 150 μL |
| Step Reagent | Inc. (min): 0:00 | Temperature: Ambient | Supplier: B. BRAUN Medical Inc. #R5000-01 |
| 50 *Deionized Water | | | Dispense type: 150 μL |
| Step type: Wash | | | |
| Step Reagent | Inc. (min): 1:00 | Temperature: Ambient | Supplier: Leica Microsystems |
| 51 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 52 *BondWashSolution | | | Cat# AR9590 |
| Step type: Wash | | | Dispense type: 150 μL |
| Step Reagent | Inc. (min): 0:00 | Temperature: Ambient | Supplier: B. BRAUN Medical Inc. #R5000-01 |
| 53 *Deionized Water | | | Dispense type: 150 μL |
| Step type: Wash | | | |

TABLE E

| Step Reagent | Inc. (min): 30:00 | Temperature: Ambient | Supplier: SantaCruze, (Cat# SC-21248; |
|---|---|---|---|
| 1 Goat anti- | | | Lot# A2313) |
| human | | | Dispense type: 150 μL |
| NESTIN | | | |
| Step type: Reagent | | | |
| Step Reagent | Inc. (min): 2:00 | Temperature: Ambient | Supplier: Leica Microsystems |
| 2 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 3 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 4 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 5 *BondWashSolution | | | Cat# AR9590 |
| Step type: Wash | | | Dispense type: 150 μL |
| Step Reagent | Inc. (min): 8:00 | Temperature: Ambient | Supplier: Vector Lab (Cat# BA-5000) |
| 6 Rabbit anti- | | | Dispense type: 150 0L |
| goat | | | |
| secondary | | | |
| antibody | | | |
| Step type: Reagent | | | |
| Step Reagent | Inc. (min): 2:00 | Temperature: Ambient | Supplier: Leica Microsystems |
| 7 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 8 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 9 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 10 *BondWashSolution | | | Cat# AR9590 |
| Step type: Wash | | | Dispense type: 150 μL |
| Step Reagent | Inc. (min): 8:00 | Temperature: Ambient | Supplier: Bond Polymer Refine Detection kit |
| 11 *Polymer | | | Cat# DS9800 |
| Step type: Reagent | | | Dispense type: 150 μL |
| Step Reagent | Inc. (min): 2:00 | Temperature: Ambient | Supplier: Leica Microsystems |
| 12 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 13 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 14 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 15 *BondWashSolution | | | Cat# AR9590 |
| Step type: Wash | | | Dispense type: 150 μL |
| Step Reagent | Inc. (min): 10:00 | Temperature: Ambient | Supplier: MSKCC |
| 16 Compound | | | Dispense type: 150 μL |
| Step type: Reagent | | | |
| Step Reagent | Inc. (min): 2:00 | Temperature: Ambient | Supplier: Leica Microsystems |
| 17 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 18 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 19 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 20 *BondWashSolution | | | Cat# AR9590 |
| Step type: Wash | | | Dispense type: 150 μL |
| Step Reagent | Inc. (min): 0:00 | Temperature: 100° C. | Supplier: Perkin Elmer |
| 21 AR9 Buffer | | | Cat# AR900250ML |
| Step type: Reagent | | | Dispense type: 150 μL |
| Step Reagent | Inc. (min): 20:00 | Temperature: 100° C. | Supplier: Perkin Elmer |
| 22 AR9 Buffer | | | Cat# AR900250ML |
| Step type: Reagent | | | Dispense type: 150 μL |
| Step Reagent | Inc. (min): 0:00 | Temperature: 100 | Supplier: Perkin Elmer |
| 23 AR9 Buffer | | | Cat# AR900250ML |
| Step type: Reagent | | | Dispense type: 150 μL |
| | | | Dispense type: 150 μL |
| Step Reagent | Cat#AR900250ML | | Supplier: Perkin Elmer |
| 24 AR9 Buffer | Inc. (min): 2:00 | Temperature: Ambient | |
| Step type: Reagent | | | |
| Step Reagent | Inc. (min): 0:00 | Temperature: Ambient | Supplier: Perkin Elmer |
| 25 AR9 Buffer | | | Cat# AR900250ML |
| Step type: Reagent | | | Dispense type: 150 μL |
| Step Reagent | Inc. (min): 0:00 | Temperature: Ambient | Supplier: Leica Microsystems |
| 26 *BondWashSolution | | | Cat# AR9590 |
| Step type: Wash | | | Dispense type: 150 μL |
| Step Reagent | Inc. (min): 10:00 | Temperature: Ambient | Supplier: Vector Lab |
| 27 Normal horse serum | | | Cat# MP-7402 kit |
| Step type: Reagent | | | Dispense type: 150 μL |
| 28 Mouse | Inc. (min): 30:00 | Temperature: Ambient | Supplier: Creative Biolabs (2D12.5) |
| anti- | | | Cat# PABW-133 |
| DOTA | | | Dispense type: 150 μL |
| antibody | | | |
| 2D12.5 | | | |
| Step type: Reagent | | | |
| Step Reagent | Inc. (min): 2:00 | Temperature: Ambient | Supplier: Leica Microsystems |
| 29 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 30 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 31 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 32 *BondWashSolution | | | Cat# AR9590 |
| Step type: Wash | | | Dispense type: 150 μL |
| Step Reagent | Inc. (min): 20:00 | Temperature: Ambient | Supplier: Vector Lab |
| 33 Horse anti-mouse | | | Cat# MP-7402 kit |
| IgG | | | Dispense type: 150 μL |
| Step type: Reagent | | | |
| Step Reagent | Inc. (min): 2:00 | Temperature: Ambient | Supplier: Leica Microsystems |
| 34 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 35 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 36 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 37 *BondWashSolution | | | Cat# AR9590 |
| Step type: Wash | | | Dispense type: 150 μL |
| Step Reagent | Inc. (min): 8:00 | Temperature: Ambient | Supplier: Bond Polymer Refine Detection kit |
| 38 *Peroxide Block | | | Cat# DS9800 |
| Step type: Reagent | | | Dispense type: 150 μL |
| Step Reagent | Inc. (min): 0:00 | Temperature: Ambient | Supplier: Leica Microsystems |
| 39 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 40 *BondWashSolution | | | Cat# AR9590 |
| Step type: Wash | | | Dispense type: Open |
| Step Reagent | Inc. (min): 0:00 | Temperature: Ambient | Supplier: Leica Microsystems |
| 41 *BondWashSolution | | | Cat# AR9590 |
| Step type: Wash | | | Dispense type: 150 μL |
| Step Reagent | Inc. (min): 0:00 | Temperature: Ambient | Supplier: B. BRAUN Medical Inc. #R5000-01 |
| 42 *Deionized Water | | | Dispense type: 150 μL |
| Step type: Reagent | | | |
| Step Reagent | Inc. (min): 0:00 | Temperature: Ambient | Supplier: B. BRAUN Medical Inc. #R5000-01 |
| 43 *Deionized Water | | | Dispense type: 150 μL |
| Step type: Wash | | | |
| Step Reagent | Inc. (min): 2:00 | Temperature: Ambient | Supplier: Bond Polymer Refine Detection kit |
| 44 *MixedDABRefine | | | Cat# DS9800 |
| Step type: Reagent | | | Dispense type: 150 μL |
| Step Reagent | Inc. (min): 5:00 | Temperature: Ambient | Supplier: Bond Polymer Refine Detection kit |
| 45 *MixedDABRefine | | | Cat# DS9800 |
| Step type: Reagent | | | Dispense type: 150 μL |
| Step Reagent | Inc. (min): 0:00 | Temperature: Ambient | Supplier: B. BRAUN Medical Inc. #R5000-01 |
| 46 *Deionized Water | | | Dispense type: 150 μL |
| Step type: Wash | | | |
| Step Reagent | Inc. (min): 0:00 | Temperature: Ambient | Supplier: B. BRAUN Medical Inc. #R5000-01 |
| 47 *Deionized Water | | | Dispense type: 150 μL |
| Step type: Wash | | | |
| Step Reagent | Inc. (min): 0:00 | Temperature: Ambient | Supplier: B. BRAUN Medical Inc. #R5000-01 |
| 48 *Deionized Water | | | Dispense type: 150 μL |
| Step type: Wash | | | |
| Step Reagent | Inc. (min): 10:00 | Temperature: Ambient | Supplier: Bond Polymer Refine Detection kit |
| 49 *Hematoxylin | | | Cat# DS9800 |
| Step type: Reagent | | | Dispense type: 150 μL |
| Step Reagent | Inc. (min): 0:00 | Temperature: Ambient | Supplier: B. BRAUN Medical Inc. #R5000-01 |
| 50 *Deionized Water | | | Dispense type: 150 μL |
| Step type: Wash | | | |
| Step Reagent | Inc. (min): 1:00 | Temperature: Ambient | Supplier: Leica Microsystems |
| 51 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 52 *BondWashSolution | | | Cat# AR9590 |
| Step type: Wash | | | Dispense type: 150 μL |
| Step Reagent | Inc. (min): 0:00 | Temperature: Ambient | Supplier: B. BRAUN Medical Inc. #R5000-01 |
| 53 *Deionized Water | | | Dispense type: 150 μL |
| Step type: Wash | | | |

TABLE F

| | | | |
|---|---|---|---|
| Step Reagent | Inc. (min): 30:00 | Temperature: Ambient | Supplier: SantaCruze, (Cat# SC-21248; |
| 1 Goat anti- | | | Lot# A2313) |
| human | | | Dispense type: 150 μL |
| NESTIN | | | |
| Step type: Reagent | | | |
| Step Reagent | Inc. (min): 2:00 | Temperature: Ambient | Supplier: Leica Microsystems |
| 2 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 3 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 4 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 5 *BondWashSolution | | | Cat# AR9590 |
| Step type: Wash | | | Dispense type: 150 μL |
| Step Reagent | Inc. (min): 8:00 | Temperature: Ambient | Supplier: Vector Lab (Cat# BA-5000) |
| 6 Rabbit anti- | | | Dispense type: 150 μL |
| goat | | | |
| secondary | | | |
| antibody | | | |
| Step type: Reagent | | | |
| Step Reagent | Inc. (min): 2:00 | Temperature: Ambient | Supplier: Leica Microsystems |
| 7 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 8 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 9 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 10 *BondWashSolution | | | Cat# AR9590 |
| Step type: Wash | | | Dispense type: 150 μL |
| Step Reagent | Inc. (min): 8:00 | Temperature: Ambient | Supplier: Bond Polymer Refine Detection kit |
| 11 *Polymer | | | Cat# DS9800 |
| Step type: Reagent | | | Dispense type: 150 μL |
| Step Reagent | Inc. (min): 2:00 | Temperature: Ambient | Supplier: Leica Microsystems |
| 12 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 13 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 14 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 15 *BondWashSolution | | | Cat# AR9590 |
| Step type: Wash | | | Dispense type: 150 μL |
| Step Reagent | Inc. (min): 10:00 | Temperature: Ambient | Supplier: MSKCC |
| 16 Compound | | | Dispense type: 150 μL |
| Step type: Reagent | | | |
| Step Reagent | Inc. (min): 2:00 | Temperature: Ambient | Supplier: Leica Microsystems |
| 17 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 18 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 19 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 20 *BondWashSolution | | | Cat# AR9590 |
| Step type: Wash | | | Dispense type: 150 μL |
| Step Reagent | Inc. (min): 0:00 | Temperature: 100° C. | Supplier: Perkin Elmer |
| 21 AR9 Buffer | | | Cat# AR900250ML |
| Step type: Reagent | | | Dispense type: 150 μL |
| Step Reagent | Inc. (min): 20:00 | Temperature: 100° C. | Supplier: Perkin Elmer |
| 22 AR9 Buffer | | | Cat# AR900250ML |
| Step type: Reagent | | | Dispense type: 150 μL |
| Step Reagent | Inc. (min): 0:00 | Temperature: 100 | Supplier: Perkin Elmer |
| 23 AR9 Buffer | | | Cat# AR900250ML |
| Step type: Reagent | | | Dispense type: 150 μL |
| Step Reagent | Inc. (min): 2:00 | Temperature: Ambient | Supplier: Perkin Elmer |
| 24 AR9 Buffer | | | Cat#AR900250ML |
| Step type: Reagent | | | Dispense type: 150 μL |
| Step Reagent | Inc. (min): 0:00 | Temperature: Ambient | Supplier: Perkin Elmer |
| 25 AR9 Buffer | | | Cat# AR900250ML |
| Step type: Reagent | | | Dispense type: 150 μL |
| Step Reagent | Inc. (min): 0:00 | Temperature: Ambient | Supplier: Leica Microsystems |
| 26 *BondWashSolution | | | Cat# AR9590 |
| Step type: Wash | | | Dispense type: 150 μL |
| Step Reagent | Inc. (min): 10:00 | Temperature: Ambient | Supplier: Vector Lab |
| 27 Normal horse serum | | | Cat# MP-7402 kit |
| Step type: Reagent | | | Dispense type: 150 μL |
| Step Reagent | Inc. (min): 30:00 | Temperature: Ambient | Supplier: VWR |
| 28 TBS-t | | | Cat# J640-1L |
| Step type: Reagent | | | Dispense type: 150 μL |
| Step Reagent | Inc. (min): 2:00 | Temperature: Ambient | Supplier: Leica Microsystems |
| 29 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 30 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 31 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 32 *BondWashSolution | | | Cat# AR9590 |
| Step type: Wash | | | Dispense type: 150 μL |
| Step Reagent | Inc. (min): 20:00 | Temperature: Ambient | Supplier: Vector Lab |
| 33 Horse anti-mouse | | | Cat# MP-7402 kit |
| IgG | | | Dispense type: 150 μL |
| Step type: Reagent | | | |
| Step Reagent | Inc. (min): 2:00 | Temperature: Ambient | Supplier: Leica Microsystems |
| 34 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 35 *BondWashSolution | | | Cat# AR9590 |
| Step type: Wash | | | Dispense type: 150 μL |
| Step Reagent | Inc. (min): 2:00 | Temperature: Ambient | Supplier: Leica Microsystems |
| 36 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 37 *BondWashSolution | | | Cat# AR9590 |
| Step type: Wash | | | Dispense type: 150 μL |
| Step Reagent | Inc. (min): 8:00 | Temperature: Ambient | Supplier: Bond Polymer Refine Detection kit |
| 38 *Peroxide Block | | | Cat# DS9800 |
| Step type: Reagent | | | Dispense type: 150 μL |
| Step Reagent | Inc. (min): 0:00 | Temperature: Ambient | Supplier: Leica Microsystems |
| 39 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 40 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 41 *BondWashSolution | | | Cat# AR9590 |
| Step type: Wash | | | Dispense type: 150 μL |
| Step Reagent | Inc. (min): 0:00 | Temperature: Ambient | Supplier: B. BRAUN Medical Inc. #R5000-01 |
| 42 *Deionized Water | | | Dispense type: 150 μL |
| Step type: Reagent | | | |
| Step Reagent | Inc. (min): 0:00 | Temperature: Ambient | Supplier: B. BRAUN Medical Inc. #R5000-01 |
| 43 *Deionized Water | | | Dispense type: 150 μL |
| Step type: Wash | | | |
| Step Reagent | Inc. (min): 2:00 | Temperature: Ambient | Supplier: Bond Polymer Refine Detection kit |
| 44 *MixedDABRefine | | | Cat# DS9800 |
| Step type: Reagent | | | Dispense type: 150 μL |
| Step Reagent | Inc. (min): 5:00 | Temperature: Ambient | Supplier: Bond Polymer Refine Detection kit |
| 45 *MixedDABRefine | | | Cat# DS9800 |
| Step type: Reagent | | | Dispense type: 150 μL |
| Step Reagent | Inc. (min): 0:00 | Temperature: Ambient | Supplier: B. BRAUN Medical Inc. #R5000-01 |
| 46 *Deionized Water | | | Dispense type: 150 μL |
| Step type: Wash | | | |
| Step Reagent | Inc. (min): 0:00 | Temperature: Ambient | Supplier: B. BRAUN Medical Inc. #R5000-01 |
| 47 *Deionized Water | | | Dispense type: 150 μL |
| Step type: Wash | | | |
| Step Reagent | Inc. (min): 0:00 | Temperature: Ambient | Supplier: B. BRAUN Medical Inc. #R5000-01 |
| 48 *Deionized Water | | | Dispense type: 150 μL |
| Step type: Wash | | | |
| Step Reagent | Inc. (min): 10:00 | Temperature: Ambient | Supplier: Bond Polymer Refine Detection kit |
| 49 *Hematoxylin | | | |
| Step type: Reagent | | | |
| Step Reagent | Inc. (min): 0:00 | Temperature: Ambient | Supplier: B. BRAUN Medical Inc. #R5000-01 |
| 50 *Deionized Water | | | Dispense type: 150 μL |
| Step type: Wash | | | |
| Step Reagent | Inc. (min): 1:00 | Temperature: Ambient | Supplier: Leica Microsystems |
| 51 *BondWashSolution | | | Cat# AR9590 |
| Step type: Wash | | | Dispense type: 150 μL |
| Step Reagent | Inc. (min): 0:00 | Temperature: Ambient | Supplier: Leica Microsystems |
| 52 *BondWashSolution | | | Cat# AR9590 |
| Step type: Wash | | | Dispense type: 150 μL |
| Step Reagent | Inc. (min): 0:00 | Temperature: Ambient | Supplier: B. BRAUN Medical Inc. #R5000-01 |
| 53 *Deionized Water | | | Dispense type: 150 μL |
| Step type: Wash | | | |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise. This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 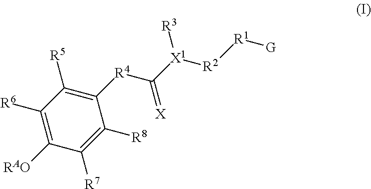
or a salt thereof, wherein:
G is a chelating moiety;
R¹ is substituted or unsubstituted alkylene, a bond, —O—, —S—, or —NR^{A}—;
R² is a bond, substituted or unsubstituted carbocyclylene, substituted or unsubstituted heterocyclylene, substituted or unsubstituted arylene, or substituted or unsubstituted heteroarylene;
X¹ is a bond or N;
X is O or S;
R³ is hydrogen, substituted or unsubstituted C₁-C₆ alkyl, or a nitrogen protecting group;
R⁴ is substituted or unsubstituted C₂-C₆ heteroaliphatic, substituted or unsubstituted C₂-C₆ alkylene, substituted or unsubstituted C₂-C₆ alkenylene, or substituted or unsubstituted C₂-C₆ alkynylene;
R⁵ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};
R^{b} is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};
R⁷ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A};
R⁸ is hydrogen, halogen, nitro, cyano, substituted or unsubstituted alkyl, substituted or unsubstituted alkenyl, substituted or unsubstituted alkynyl, substituted or unsubstituted acyl, substituted or unsubstituted carbocyclyl, substituted or unsubstituted heterocyclyl, substituted or unsubstituted aryl, substituted or unsubstituted heteroaryl, —N(R^{A})₂, or —OR^{A}; and
each occurrence of R^{A} is independently hydrogen, substituted or unsubstituted acyl, substituted or unsubstituted alkyl, substituted or unsubstituted alkenyl, substituted or unsubstituted alkynyl, substituted or unsubstituted heteroalkyl, a nitrogen protecting group when attached to a nitrogen atom, or an oxygen protecting group when attached to an oxygen atom, or two R^{A} groups are joined to form a substituted or unsubstituted heterocyclic ring.

2. The compound of claim 1, or a salt thereof, wherein R¹ is substituted or unsubstituted C₁-C₆ alkylene.

3. The compound of claim 1, or a salt thereof, wherein R¹ is unsubstituted C₁-C₆ alkylene.

4. The compound of any of claims 1-3, or a salt thereof, wherein R² is substituted or unsubstituted arylene.

5. The compound of any of claims 1-3, or a salt thereof, wherein R² is unsubstituted phenylene.

6. The compound of any of claims 1-5, or a salt thereof, wherein X is O.

7. The compound of any of claims 1-5, or a salt thereof, wherein X is S.

8. The compound of any of claims 1-7, or a salt thereof, wherein X¹ is N.

9. The compound of any of claims 1-8, or a salt thereof, wherein R³ is hydrogen.

10. The compound of any of claims 1-9, or a salt thereof, wherein R⁴ is substituted or unsubstituted C₂-C₆ heteroaliphatic, substituted or unsubstituted C₂-C₆ alkylene, or substituted or unsubstituted C₂-C₆ alkenylene.

11. The compound of any of claims 1-10, or a salt thereof, wherein R⁴ is unsubstituted C₂-C₆ heteroaliphatic, unsubstituted C₂-C₆ alkylene, or unsubstituted C₂-C₆ alkenylene.

12. The compound of any of claims 1-11, or a salt thereof, wherein R⁴ is unsubstituted C₂-C₃ heteroaliphatic, unsubstituted C₂-C₃ alkylene, or unsubstituted C₂-C₃ alkenylene.

13. The compound of any of claims 1-12, or a salt thereof, wherein R⁴ is

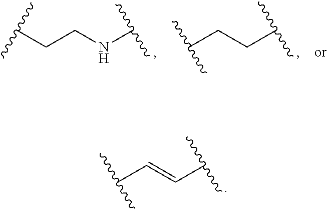

14. The compound of any of claims 1-13, or a salt thereof, wherein R⁵, R⁶, R⁷, and R⁸ are independently hydrogen, halogen, nitro, cyano, substituted or unsubstituted alkyl, substituted or unsubstituted alkenyl, substituted or unsubstituted alkynyl, substituted or unsubstituted acyl, —N(R^{A})₂, or —OR^{A}.

15. The compound of any of claims 1-14, or a salt thereof, wherein R⁵, R⁶, R⁷, and R⁸ are independently hydrogen, —N(R^{A})₂, or —OR^{A}.

16. The compound of any of claims 1-15, or a salt thereof, wherein at least one of R⁵, R⁶, R⁷, and R⁸ is —OR^{A}.

17. The compound of any of claims 1-16, or a salt thereof, wherein R⁵, R⁷, and R⁸ are each hydrogen.

18. The compound of any of claims 1-17, wherein R^{A} is hydrogen.

19. The compound of claim 1, wherein the compound is of Formula (I-a): 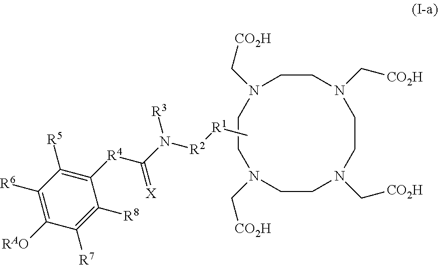
or a salt thereof.

20. The compound of claim 1, wherein the compound is of Formula (I-b): 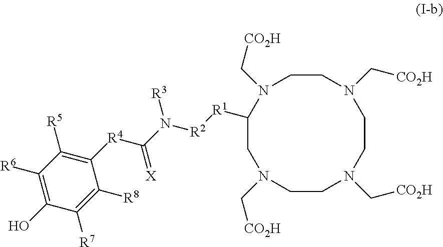
or a salt thereof.

21. The compound of claim 1, wherein the compound is of Formula (I-c): 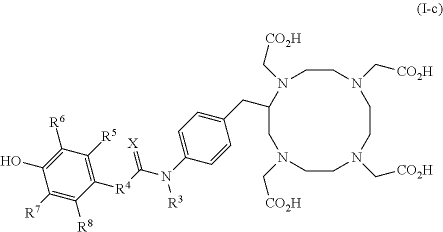
or a salt thereof.

22. The compound of claim 1, wherein the compound is of Formula (I-d): 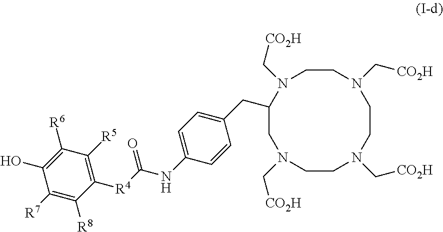
or a salt thereof.

23. The compound of claim 1, wherein the compound is of Formula (I-e): 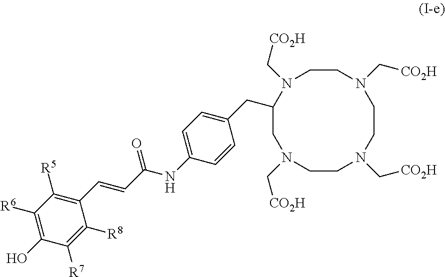
or a salt thereof.

24. The compound of claim 1, wherein the compound is of Formula (I-f): 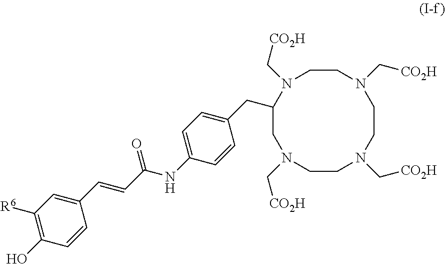
or a salt thereof.

25. The compound of claim 1, wherein the compound is of Formula (I-g): 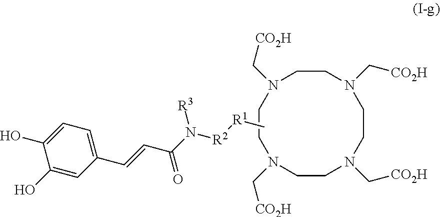
or a salt thereof.

26. The compound of claim 1, wherein the compound is of Formula (I-h): 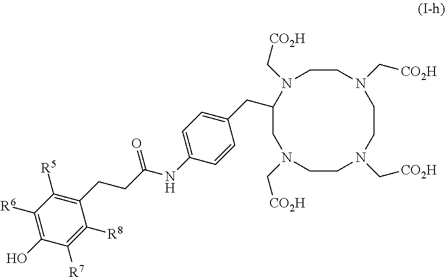
or a salt thereof.

27. The compound of claim 1, wherein the compound is of Formula (I-i): 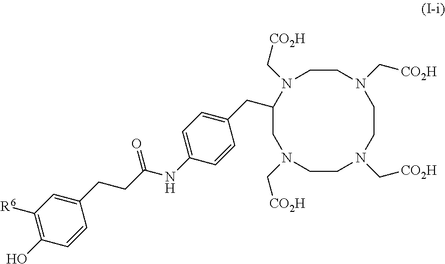
or a salt thereof.

28. The compound of claim 1, wherein the compound is of Formula (I-j): 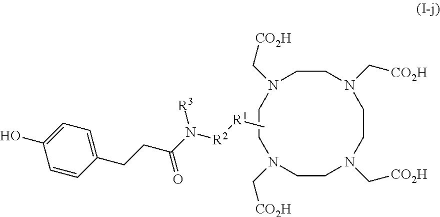
or a salt thereof.

29. The compound of claim 1, wherein the compound is of Formula (I-k): 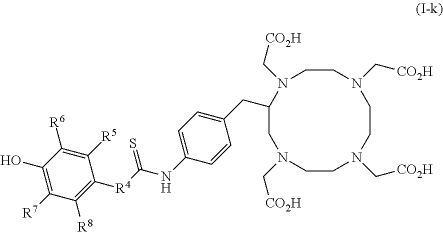
or a salt thereof.

30. The compound of claim 1, wherein the compound is of Formula (I-l): 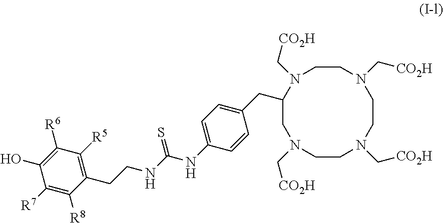
or a salt thereof.

31. The compound of claim 1, wherein the compound is of Formula (I-m): 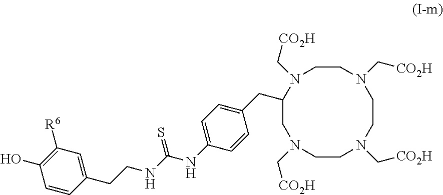
or a salt thereof.

32. The compound of claim 1, wherein the compound is of Formula (I-n): 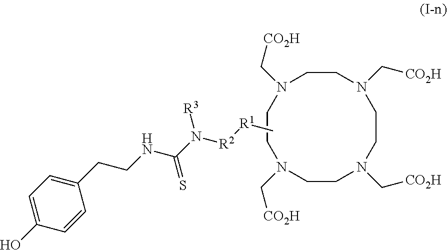
or a salt thereof.

33. The compound of claim 1, wherein the compound is of the formula: 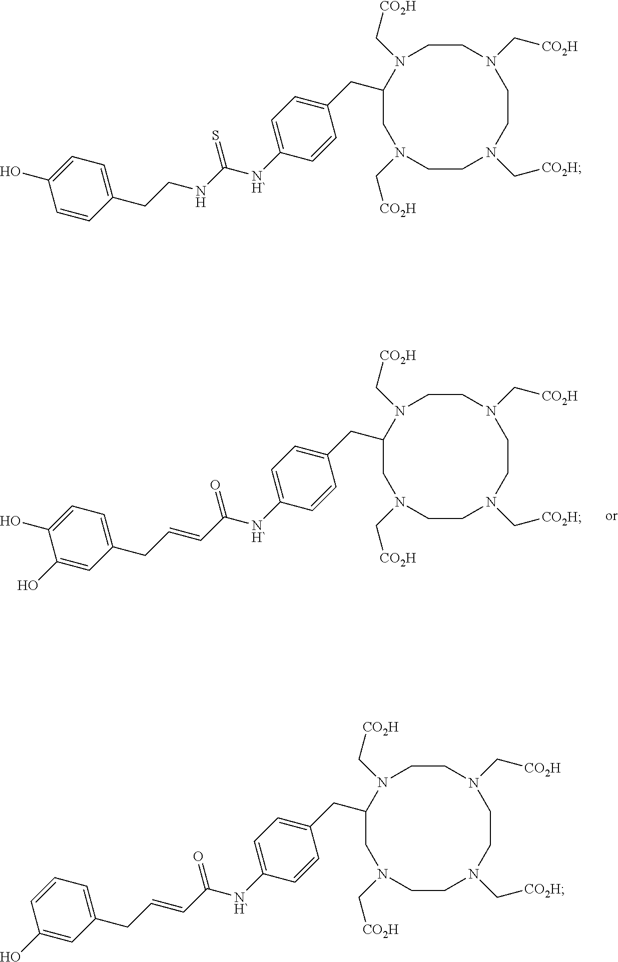
or a salt thereof.

34. A salt of the compound of any of claims 1-33.

35. The salt of claim 34, wherein the salt comprises a metal counterion.

36. The salt of claim 35, wherein the metal counterion is bismuth, lead, yttrium, cadmium, mercury, actinium, thorium, strontium, or a lanthanide.

37. The salt of claim 35 or 36, wherein the metal counterion is yttrium, praseodymium, or lutetium.

38. An yttrium (Y³⁺) salt of the compound of any of claims 1-33.

39. A praseodymium (Pr³⁺) salt of the compound of any of claims 1-33.

40. A chelate complex comprising the compound of any of claims 1-33 or the salt of any of claims 34-39.

41. The chelate complex of claim 40, wherein the chelate complex is a trivalent complex.

42. The chelate complex of claim 41, wherein the chelate complex is of the formula:

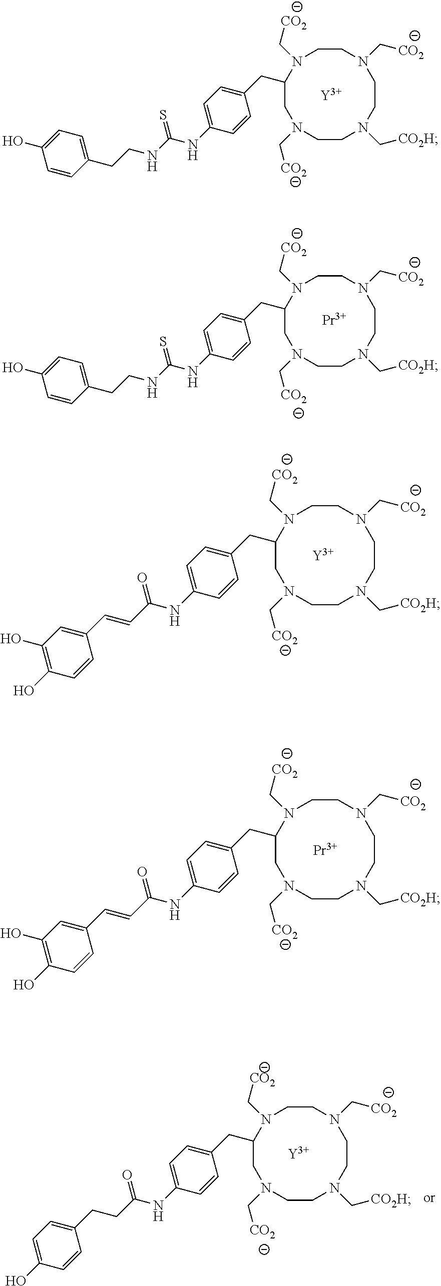

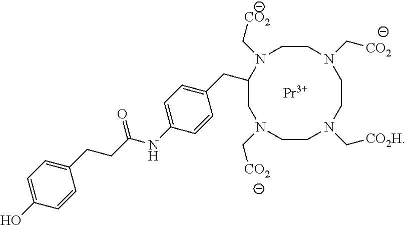

43. A method of detecting an analyte, the method comprising:
reacting an enzyme with the chelate complex of any of claims 40-42 to form an oxidized chelate complex;
contacting the oxidized chelate complex with a biological sample; and
detecting the analyte in the biological sample.

44. The method of claim 43 further comprising covalent binding of the oxidized chelate complex with the analyte.

45. The method of any of claim 43 or 44, wherein the analyte comprises a protein derived from human tissue.

46. The method of any of claims 43-45, wherein the enzyme is a peroxidase.

47. The method of any of claims 43-46, wherein the detecting comprises mass spectrometry imaging based microscopy.

48. The chelate complex of any of claims 40-42 for use in the detection of an analyte.

49. The chelate complex of claim 48, wherein the detection comprises
reacting an enzyme with the chelate complex to form an oxidized chelate complex;
contacting the oxidized chelate complex with a biological sample; and
detecting the analyte in the biological sample.

50. The chelate complex of claim 49, wherein the oxidized chelate complex covalently binds with the analyte.

51. The chelate complex of any of claims 48-50, wherein the analyte comprises a protein derived from human tissue.

52. The chelate complex of any of claims 49-51, wherein the enzyme is a peroxidase.

53. The chelate complex of any of claims 48-52, wherein the detection comprises mass spectrometry imaging based microscopy.

54. A kit comprising the chelate complex of any of claims 38-40, and instructions for using the chelate complex for detection of an analyte.

55. The kit of claim 54 further comprising an enzyme.

56. The kit of claim 55, wherein the enzyme is a peroxidase.
